(19) United States
(12) Patent Application Publication
(10) Pub. No.: US 2015/0064234 A1
(43) Pub. Date: Mar. 5, 2015

(54) 4-AMINOPYRIDINE AS A THERAPEUTIC AGENT FOR SPINAL MUSCULAR ATROPHY

(71) Applicant: Brian D. Mccabe, New York, NY (US)

(72) Inventor: Brian D. Mccabe, New York, NY (US)

(73) Assignee: THE TRUSTEES OF COLUMBIA UNIVERSITY IN THE CITY OF NEW YORK, NEW YORK, NY (US)

(21) Appl. No.: 14/388,421

(22) PCT Filed: Mar. 26, 2013
(86) PCT No.: PCT/US13/33952

§ 371 Date: Sep. 26, 2014

Related U.S. Application Data
(60) Provisional application No. 61/615,466, filed on Mar. 26, 2012.

(51) Int. Cl. A61K 31/4409; A61K 9/127

(52) U.S. Cl. CPC A61K 31/4409; A61K 9/127
USPC 424/450; 514/352; 514/357; 514/605; 514/254.07; 514/643; 514/318; 514/274; 514/278

(57) ABSTRACT

It has been discovered that pharmacological inhibition of K+ channels (using the FDA-approved broad-spectrum K+ channel antagonist 4-AP) positively benefitted smn mutant phenotypes, a result that is consistent with the defective excitability of motor circuits by their interneuron or sensory neuron inputs being a critical consequence of SMN depletion. Based on these observations, certain embodiments of the invention are directed to methods of treatment of SMA by administering therapeutically effective amounts of one or more potassium channel antagonists, including 4-aminopyridine, 4-(dimethylamino)pyridine, 4-(methylamino)pyridine, and 4-(aminomethyl)pyridine. Other embodiments are directed to new pharmaceutical formulations comprising two or more potassium channel antagonists.

### CROSS-REFERENCE TO RELATED APPLICATIONS

The present application claims priority to U.S. Provisional Application. No. 61/615,466 filed Mar. 15, 2008 and U.S. Provisional Application. No. 61/057,190 filed on Mar. 26, 2012, the entire contents of which are hereby incorporated by reference as if fully set forth herein, under 35 U.S.C. §119(e).

### STATEMENT OF GOVERNMENTAL INTEREST

This invention was made with Government support under Contract No. W81XWH-08-1-0009 awarded by the Department of Defense. The Government has certain rights in the invention.

### BACKGROUND OF THE INVENTION

Spinal muscular atrophy (SMA) is a lethal human disease characterized by motor neuron dysfunction and muscle deterioration due to depletion of the ubiquitous Survival Motor Neuron (SMN) protein. SMA is an autosomal recessive disease characterized by degeneration of motor neurons in the anterior horn of the spinal cord, leading to muscular paralysis and atrophy. SMA is traditionally categorized into three types, according to the age and severity: Infantile SMA-Type 1 or Werdnig-Hoffmann disease (generally 0-6 months) is the most severe form, and manifests in the first year of life resulting in an inability to ever maintain an independent sitting position. Intermediate SMA-Type 2 (generally 7-18 months) describes those children who are never able to stand and walk, but who are able to maintain a sitting position at least some time in their life. The onset of weakness is usually recognized sometime between 6 and 18 months. Juvenile SMA-Type 3 or Kugelberg-Welander disease (generally >18 months describes those who are able to walk at some time. Adult SMA-Type 4 is associated with weakness that usually begins in late adolescence in tongue, hands, or feet then progresses to other areas of the body. The course of disease is much slower and has little or no impact on life expectancy. Additionally, for prenatal onset of very severe symptoms of SMA and early neonatal death results, SMA is categorized as type 0 (Eur J Paediatr Neurol 1999; 3:49-51; Lancet 1995; 346:1162; Neuromuscul Disord 1992; 2:423-428). SMA occurs in approximately 1 in 6000-10000 live births and has a carrier frequency of 1 in 50. It is the second most common autosomal recessive inherited disorder in humans and the most common genetic cause of infant mortality (Semin Neurol 1998; 18:19-26).

Linkage mapping identified the Survival of Motor Neuron (SMN) gene as the genetic locus of SMA (Lefebvre et al., Cell 80, 1-5). In humans, two nearly identical SMN genes (SMN1 and SMN2) exist on chromosome 5q13. Deletions or mutations within SMN1 but not the SMN2 gene cause all forms of proximal SMA (Lefebvre et al., Cell 80, 1-5). SMN1 encodes a ubiquitously expressed 38 kDa SMN protein that is necessary for snRNP assembly, an essential process for cell survival (Wan, L., et al. 2005. Mol. Cell. Biol. 25:5543-5551). A nearly identical copy of the gene, SMN2, fails to compensate for the loss of SMN1 because of exon 7 skipping, producing an unstable truncated protein, SMN.DELTA.7 (Lorson, C. L., et al. 1998. Nat. Genet. 19:63-66; Lefebvre et al., 1995; Burghes and Beattie, 2009).

SMN1 and SMN2 differ by a critical C to T substitution at position 6 of exon 7 (C6U in transcript of SMN2) (Lorson, C. L., et al. 1999. Proc. Natl. Acad. Sci. USA 96:6307-6311; Monani, U. R., et al. 1999. Hum. Mol. Genet. 8:1177-1183). C6U does not change the coding sequence, but is sufficient to cause exon 7 skipping in SMN1. Therefore SMA is caused by low levels of SMN1 as opposed to the complete loss of SMN1 (Burghes and Beattie, 2009). SMN is a multifunctional protein that has been implicated in a variety of cellular processes linked to RNA metabolism (Pellizzoni, 2007).

There is no effective drug treatment for SMA, therefore there is a great need for such a treatment.

### SUMMARY OF THE INVENTION

Certain embodiments of the invention are directed to methods comprising, identifying a subject who has spinal muscular atrophy, and administering to the subject a therapeutically effective amount of a K+ channel antagonist, including a broad-based K+ channel antagonist, and antagonists selected from the group comprising 4-aminopyridine, 4-(dimethylamino)pyridine, 4-(methylamino)pyridine, and 4-(aminomethyl)pyridine. Other K+ channel antagonists for us in the present methods include dofetilide, sotalol, ibutilide), Azimilide, Bretylium, Clofilium, E-4031, Nifekalant, Tedisamil, and Sematilide. In an embodiment the therapeutically effective amount is an amount ranging from about 0.5 mg to 100 mg per administration and the antagonist is administered from one to three times per day.

Other embodiments are directed to pharmaceutical formulations, comprising 4-AP and one or more of the enumerated therapeutic agents, preferably formulated for delivery across the blood brain barrier or for administration directly into the epidural venous plexus, brain, spinal column or cerebrospinal fluid. The Present methods can be used to treat any form of SMA: types 1, 2 or 3.

### BRIEF DESCRIPTION OF THE DRAWINGS

The present invention is illustrated by way of example, and not by way of limitation, in the following FIGS.

FIG. 1. smn mutants have reduced muscle size, decreased locomotion, defective motor rhythm and aberrant neuromuscular junction (NMJ) neurotransmitter release. A-B. Sample images of muscles from segment A3 of control (A) and SMNX7 mutant (B) third instar larvae labeled with TRITC phallodin show a reduction muscle surface area (C) that is fully rescued by ubiquitous expression of UAS-flag-SMN driven by Da-Gal4 (genotype: Da-Ga14/UAS::flagSMN; SMNX7/SMNX7). D-F. 10 sample superimposed 60 second larval locomotion path traces from control (D) and SMNX7 mutants (E). smn mutant larvae have reduced velocity compared to controls that corrected by ubiquitous expression of transgenic SMN (F). G-I. Recordings from muscle 6 in segment A1 of semi-intact larval preparations where the brain, ventral nerve cord and motor neurons are intact. Control larva produce a regular motor rhythm with periodic bursting activity corresponding to peristaltic muscle contractions (G). In contrast, smn mutant larvae have an irregular motor pattern with short and uncoordinated bursts (H) as shown by an increase in the average inter-spike interval (I) that is rescued by ubiquitous expression of SMN. J-L. Representative traces recorded from muscle 6 of segment A3 in control (J) and SMNX7 mutant (K) larva. SMNX7 mutants have increased evoked Excitatory Post-Synaptic Potential (eEPSP) amplitude than controls (K). This increase is corrected by ubiquitous expression of SMN (L). Error bars represent the standard error of the mean. **=p<0.01, ***=p<0.001, significance calculated versus control. Supplementary Figure S1 shows that SMNX7 mutants have <6% SMN protein levels compared to controls.

FIG. 2. SMN expression is required in neurons but not muscles to rescue smn mutants. A-D. Sample images of muscles from segment A3 of (A) control, (B) SMNX7 mutant, (C) SMNX7 mutants with transgenic SMN expression*n only in muscles (G14-Gal4/UAS::flagSMN; SMNX7/SMNX7) or (D) neurons (nsyb-Gal4/UAS::flagSMN; SMNX7/SMNX7). Restoration of SMN expression in muscles has no effect on muscle size however restoration in neurons fully rescues muscle surface area. E-H. Quantification of muscle surface area (E), locomotion (F), motor rhythm (G) and NMJ eEPSP amplitude (H) normalized to controls. Expression of transgenic SMN in neurons rescues all of smn mutant phenotypes while expression in muscles does not. Error bars represent the standard error of the mean. **=p<0.01, ***=p<0.001, significance calculated versus control except where indicated. FIG.

FIG. 3. SMN expression is required in cholinergic neurons and not motor neurons. A-D. Representative traces of control (A), SMNX7 mutant (B), transgenic SMN expressed in the motor neurons of smn mutants (0K371-Gal4/UAS::SMN; SMNX7/SMNX7) (C), transgenic SMN expressed in the cholinergic neurons of smn mutants (Cha-Gal4/UAS::SMN; SMNX7/SMNX7) (D). Expression of transgenic SMN in motor neurons does not restore normal neurotransmitter release in smn mutants however expression of SMN on cholinergic neurons restores normal eEPSP amplitude. E-F. Quantification of muscle surface area (E), locomotion (F), motor rhythm (G) and NMJ eEPSP amplitude (H) normalized to controls. Expression of transgenic SMN in the motor neurons of smn mutants with OK371-Gal4 or OK6-Gal4 or in GABAergic neurons with GAD1-Gal4 does not rescue any phenotype. In contrast, expression of transgenic SMN cholinergic neurons with Cha-Gal4 fully rescues muscle size, locomotor velocity and central motor rhythm and restores normal eEPSP amplitude at the NMJ (D). **=p<0.01, ***=p<0.001, significance calculated versus control.

FIG. 4. SMN is required in both proprioceptive and central cholinergic neurons. A. Expression of pattern cholinergic neuron Gal4 lines (dotted). Cha-Gal4 is expressed in both central and sensory cholinergic neurons. Clh201-Gal4 in only expressed in md and es sensory neurons. 1003.3-Gal4, ppk-Gal4 and ppk-Gal4 are expressed in subsets of md, es or ch sensory neurons. Diagonal hatchlines indicate the ability to rescue of smn mutant phenotypes. B, C. UAS::CD8-GFP labeling the axons of bd and type I md sensory neurons with NP2225-Gal4 in the ventral nerve cord of wild-type (B) or SMNX7 mutants (C). Sensory axons project normally into the CNS in smn mutants. D-G. Quantification of muscle surface area (D), locomotion (E), motor rhythm (F) and NMJ eEPSP amplitude (G) normalized to controls (genotype: Gal4/UAS::flagSMN; SMNX7/SMNX7). Expression of transgenic SMN in both central and sensory cholinergic neurons in smn mutants with Cha-Gal4 fully rescues all phenotypes. Restoration of SMN all sensory neurons increases muscle size and fully rescues motor rhythm and neurotransmitter release at the NMJ but does not rescue locomotion similar to restoring SMN in proprioceptive type I md neurons and bd neurons with NP2225-Gal4. Restoration of SMN in type II, III or IV md neurons, es neurons or ch neurons with 1003.3-Gal4 or ppk-Gal4 does not rescue any smn mutant phenotype. Scale bar=10 μm. *=p<0.05, **=p<0.01, ***=p<0.001, significance calculated versus control except where indicated.

FIG. 5. Restoration of SMN after embryogenesis rescues smn mutants. A. Schematic of transgenic SMN induction in the nervous system. RU486 is required for the activation of transgene induction by geneswitch Gal4. Elav::geneswitch/UAS::flagSMN; SMNX7/SMNX7 larva were transferred to either vehicle media or RU486 containing media immediately after hatching, 48 hours after hatching or 96 hours after hatching. B. Representative traces recorded from smn mutants that were cultured on either vehicle media or RU486 media 0, 48 or 96 hours after hatching. Induction of SMN at every each time-point fully restored normal eEPSP amplitude. C-F. Quantification of muscle surface area (C), locomotion (D), motor rhythm (E) and NMJ eEPSP amplitude (F) normalized to controls. Muscle size, locomotion and motor rhythm is fully rescued if transgenic SMN is induced immediately after hatching, but if SMN induction is delayed rescue is incomplete. In contrast, induction of SMN for only 48 hours is sufficient to completely restore normal neurotransmitter release the NMJ. Error bars represent the standard error of the mean. *=p<0.05, **=p<0.01, ***=p<0.001, significance calculated versus control except where indicated.

FIG. 6. Inhibiting cholinergic neuron activity mimics smn mutant phenotypes. A. Representative traces recorded from the NMJ of control or UAS-human Kir2.1 or UASPLTXII expressed in cholinergic neurons with Cha-Gal4. Inhibiting cholinergic neuron excitability with Kir2.1 or neurotransmitter release with PLTXII increases neurotransmitter release from motor neurons. B. Expression of Kir2.1 or PLTX in cholinergic neurons disrupts rhythmic motor activity. C-F. Quantification of muscle surface area (C), locomotion (D), motor rhythm (E) and NMJ eEPSP amplitude (F) normalized to controls. Expression of Kir2.1 or PLTXII in cholinergic neurons does not alter muscle size but does reduce locomotor speed, disrupt motor rhythm and increase the amplitude of evoked neurotransmitter release from motor neurons. *=p<0.05, ***=p<0.001, significance calculated versus control except where indicated.

FIG. 7. Genetic or pharmacological inhibition of K+ channels ameliorates smn mutant phenotypes. A-C. Locomotion path traces from (A) control, (B) smn mutants and (C) smn mutants expressing a UAS dominant negative Shaker K+ channel (UAS-SDN) in cholinergic neurons with Cha-Gal4. Expressing SDN increases rescues the locomotion of smn mutants. D-G. Quantification of muscle surface area (D), locomotion (E), motor rhythm (F) and NMJ eEPSP amplitude (G) normalized to controls. Expression of SDN in cholinergic neurons with Cha-Gal4 restores muscle size (D), locomotion (E), motor rhythm (F) and NMJ neurotransmitter release (G) of smn mutants to control levels. Addition of 2 mM 4-Aminopyridine (4-AP) to culture media throughout larval development does not alter muscle size in control animals but increases the muscle size of smn mutants (D). 4-AP administration inhibits locomotion, motor rhythm and neurotransmitter release in control animals. Administration of 4-AP to smn mutants corrects locomotion (E) and neurotransmitter release at the NMJ (G) to levels not significantly different from control 4-AP treated animals, and substantially corrects defects in motor rhythm (F). *=p<0.05, **=p<0.01, ***=p<0.001, significance calculated versus control except where indicated.

FIG. 8 shows that SMNX7 mutants have <6% SMN protein levels compared to controls.

FIG. 9 A. NMJ mEPSP amplitude of smnX7 mutants is similar to wildtype (WT) controls. B. NMJ mEPSP frequency is increased in smnX7 mutants compared to controls C. NMJ quantal content is increased in smnX7 mutants compared to controls. D. smnX7 heterozygous mutants have similar NMJ eEPSP amplitude to controls. Transallelic combinations of smnX7 with smn73Ao or smnE33 have increased NMJ eEPSP amplitude similar to smnX7 homozygous mutants. E. Representative images of NMJ synaptic terminals at muscle 4 of segment A3 of third instar smnX7 heterozygous and smnX7 homozygous mutant larvae stained with anti-CSP (green) to label the presynapse and anti-hrp (red) to label the neuronal membrane. Scale bar=20 μm. F. Quantification of bouton number no change of bouton numbers in heterozygous vs. homozygous smn mutants. Error bars represent the standard error of the mean. *=p<0.05 **=p<0.01, ***=p<0.001, significance calculated versus controls.

### DETAILED DESCRIPTION

The present invention is based on the discovery that SMN must be restored in both proprioceptive neurons and cholinergic interneurons in order to rescue smn mutant phenotypes. It was further discovered that increasing the excitability of central cholinergic neurons in an animal model of SMA increased motor network activity and altered smn mutant phenotypes. Experiments showed that pharmacological inhibition of K+ channels (using the FDA-approved broad-spectrum K+ channel antagonist 4-AP) positively benefitted smn mutant phenotypes, a result that is consistent with the defective excitability of motor circuits by their interneuron or sensory neuron inputs being a critical consequence of SMN depletion. Based on these observations, certain embodiments of the invention are directed to methods of treatment of SMA by administering therapeutically effective amounts of one or more potassium channel antagonists, particularly 4-aminopyridine (hereinafter as 4-AP), 4-(dimethylamino)pyridine, 4-(methylamino)pyridine, and 4-(aminomethyl)pyridine, herein collectively “the therapeutic agents.” Other embodiments are directed to new pharmaceutical formulations comprising two or more potassium channel antagonists.

### Overview

Locomotion depends upon the coordinated activity of neuronal networks. It has been hypothesized that the chronic dysfunction of neuronal circuits may ultimately lead to degeneration of neurons within the network, both exacerbating the damage and masking the primary cause of the disorder (Palop and Mucke, 2010). SMA is the most common inherited cause of infant mortality (Pearn, 1978), and it is both recessive and monogenic. SMA is characterized by motor neuron functional alterations and degeneration.

Recent studies of another neurodegenerative disease ALS in mouse models have identified contributions of other spinal cord cells such as astrocytes to disease pathology, suggesting that interactions between motor neurons and other partner cells may be an important contributing factor to motor neuron disease (Ilieva et al., 2009).

SMN is ubiquitously expressed and highly conserved across evolution with orthologs found in mouse, zebrafish, fruit flies, nematodes and yeast (Schmid and DiDonato, 2007). In genetic models, complete removal of all SMN protein results in loss of cell viability. In contrast, the reduced level of SMN found in SMA patients does not appear to significantly perturb the majority of organ systems (Crawford and Pardo, 1996). However, SMA patients develop motor problems and muscle weakness, with the proximal limb and trunk muscles stereotypically the most severely affected, progressing eventually to respiratory insufficiency and death (Swoboda et al., 2005). Postmortem studies show SMA patients have pathologically abnormal motor neurons and evidence of motor neuron loss (Simic, 2008), however it is currently unclear if this is the primary origin of motor system dysfunction or a terminal consequence.

Much of the research on SMA has been done in mice using the SMA mouse model SMN-Δ7 in which, there is a profound early impairment of motor behavior well before the loss of motor neurons occurs (Le et al., 2005, Park et al., 2010a). Most terminals of the neuromuscular junctions (NMJ) are innervated though some have structural abnormalities (Kariya et al., 2008; Kong et al., 2009; Ling et al., 2011; McGovern et al., 2008), and NMJ neurotransmission is aberrant in these mutants with a ˜50% reduction in quantal content (Kariya et al., 2008; Kong et al., 2009). Nonetheless, these NMJ terminals still produce normal muscle twitch tension (Ling et al., 2010).

Recently, in addition to the motor neuron defects, pronounced early deficits of spinal reflexes and reduced numbers of proprioceptive synaptic inputs onto motor neurons have been described in SMN-Δ7 mice, although functional contribution of these changes to the SMA phenotype is not yet known (Ling et al., 2010; Mentis et al., 2011).

The studies herein use the Drosophila SMN Mutant Model to study the neurocircuitry and physiology of the central sensory neurons, peripheral sensory neurons, and motor neurons in the pathway associated with SMA.

### Drosophila SMN Mutant Model

Drosophila smn mutants that have reduced muscle size and defective locomotion, motor rhythm and motor neuron neurotransmission; were exploited to determine the essential cellular site and requirement for SMN in the motor system. In Drosophila, motor neurons are exclusively glutamatergic while both peripheral sensory neurons and the majority of excitatory interneurons are cholinergic Baines, 2006; Salvaterra and Kitamoto, 2001). Robust phenotypic rescue of Drosophila smn mutants was produced by genetic inhibition of Voltage Gated Potassium Channels (Kv channels) which increases the amplitude and duration of synaptic neurotransmitter release. Even though motor neurons and peripheral sensory neurons have different neurotransmitters in Drosophila compared to mammals, the effect of potassium channel activators is the same in that it increase neurotransmitter release and hence action potentials.

Proprioceptive neurons provide essential inputs to motor circuits (Hughes and Thomas, 2007) and cholinergic interneurons are critical for Drosophila CNS function (Kitamoto et al., 2000), including synaptic output onto motor neurons (Baines et al., 2001). Restoration of SMN after the completion of nervous system development is sufficient to rescue SMN-dependent phenotypes, arguing that is not the connectivity but rather the function of motor circuits that is disrupted by the depletion of SMN. Two lines of evidence further support this. Firstly, inhibiting the activity of cholinergic neurons can mimic a number of smn mutant phenotypes including non-autonomous effects on motor neurons. Secondly, increasing the excitability of motor circuits through K+ channel inhibition can rescue smn mutant defects. The present results demonstrate that depletion of SMN in Drosophila causes the dysfunction of a select subset of neurons in the motor circuit which consequently perturb the activity of other networked components of the motor system, such as motor neurons and muscles. These findings establish the Drosophila model of SMA as a paradigm for a neurological disease induced by neuronal circuit dysfunction.

### Summary of Results

Using previously described loss-of-function smn mutants (Chan et al., 2003; Chang et al., 2008; Rajendra et al., 2007), it was (1) confirmed that depletion of SMN in Drosophila resulted in reduced muscle growth and defective locomotion similar to SMA phenotypes and (2) this was accompanied by aberrant rhythmic motor output and neuromuscular junction neurotransmission. Surprisingly, none of these defects could be rescued in Drosophila smn mutants by transgenic restoration of SMN in either the muscles or motor neurons. Rather, it has now been discovered that SMN must be restored in both proprioceptive neurons and cholinergic interneurons in order to rescue smn mutant phenotypes. This discovery shows that the disruption of motor neurons and muscles is a secondary consequence of a primary dysfunction of sensory-motor network activity and further, that genetic or pharmacological manipulation of sensory neurons to increase motor circuit excitability positively benefits smn mutant phenotypes. xx

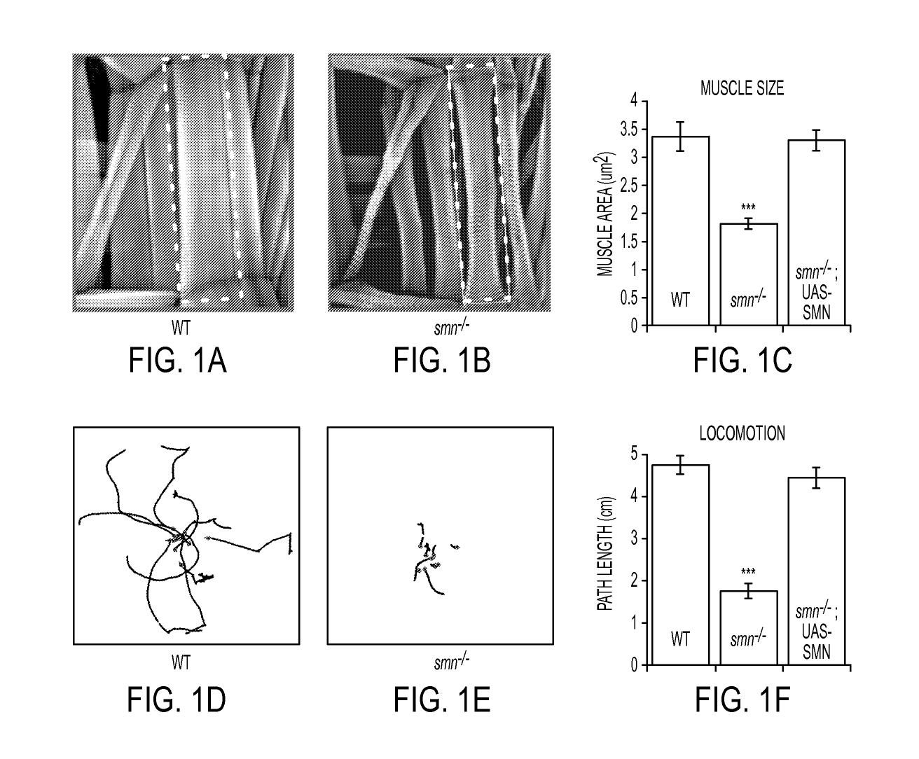

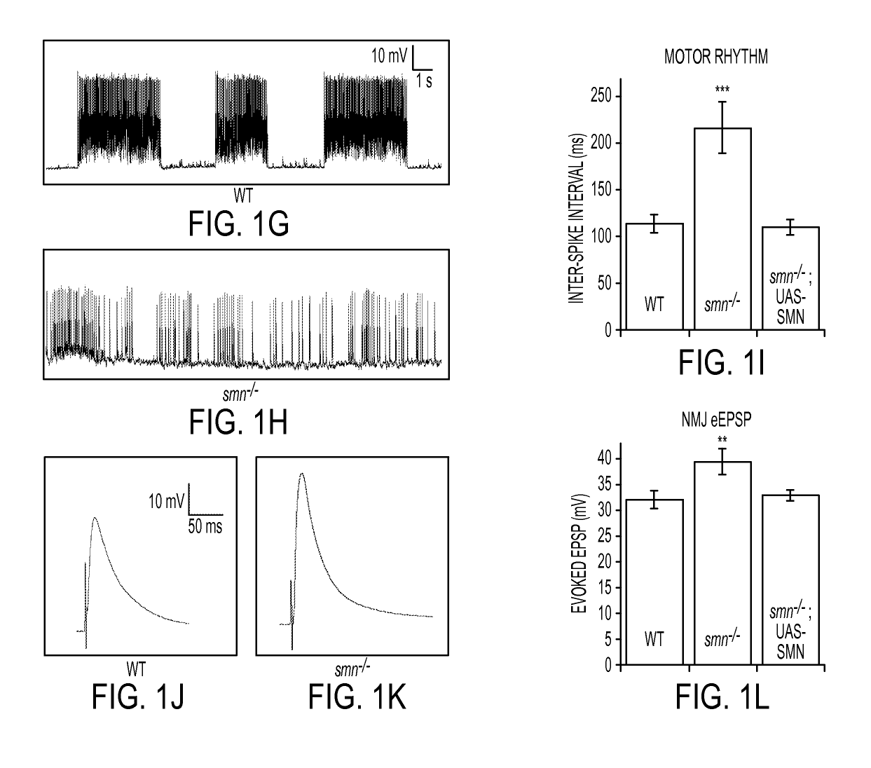

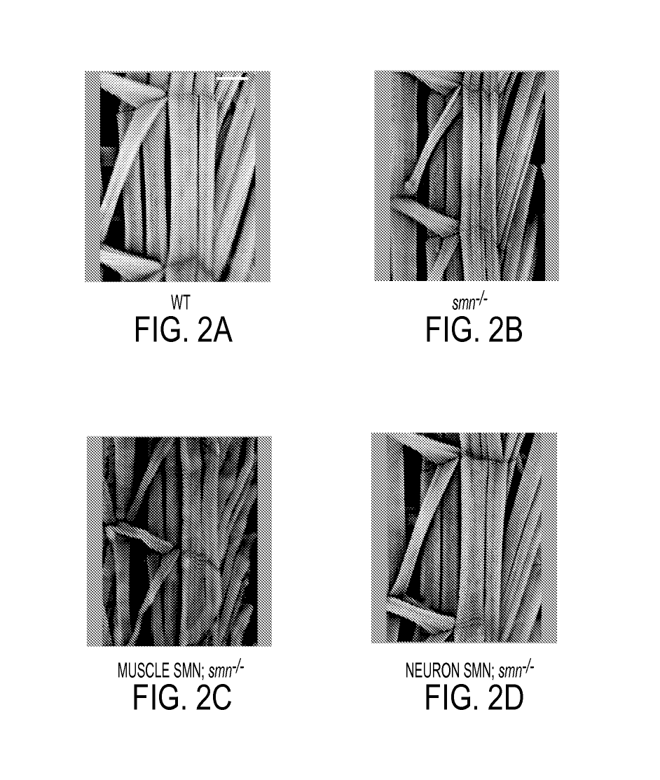

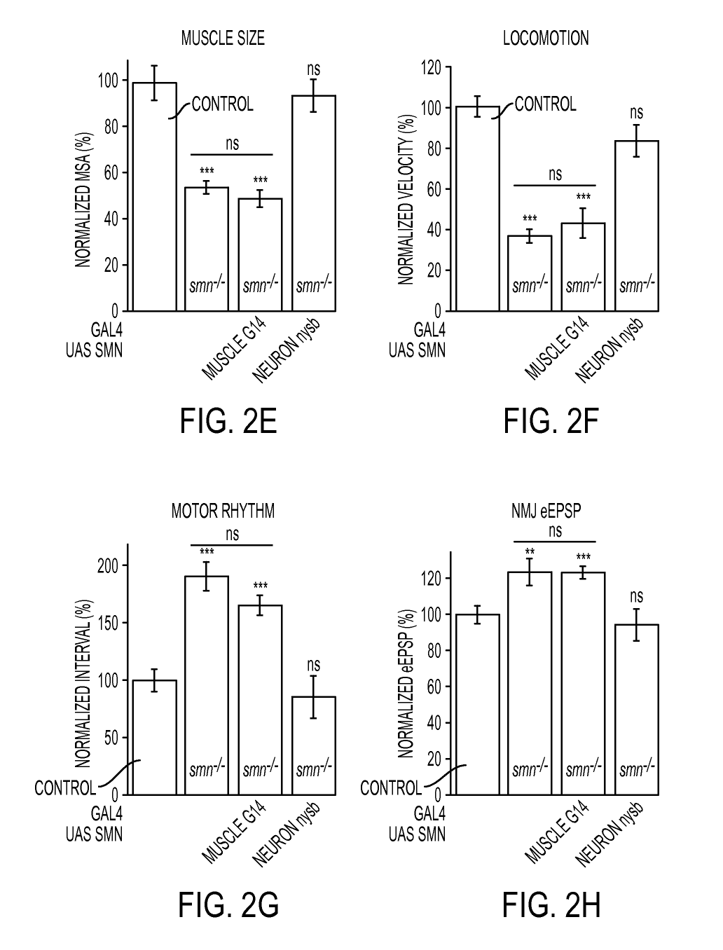

The results in Example 1 validate the Drosophila model and show also that Drosophila smn mutants have increased NMJ evoked neurotransmitter release that is accompanied by defects of muscle growth, locomotion and motor rhythm. (FIGS. 1 and 2).

The results in Example 2 shows that in contrast to muscle restoration of SMN, pan-neuronal restoration of SMN fully rescued the muscle surface area of smn mutants to control levels (FIG. 2 B,D,E) and also completely restored their locomotor velocity, rhythmic motor output and NMJ eEPSP amplitudes (FIG. FIG. 2F-H). These results showed that the defects of muscle growth in smn mutant larvae are due to a non-autonomous requirement for normal SMN levels in the nervous system rather than in muscle fibers themselves.

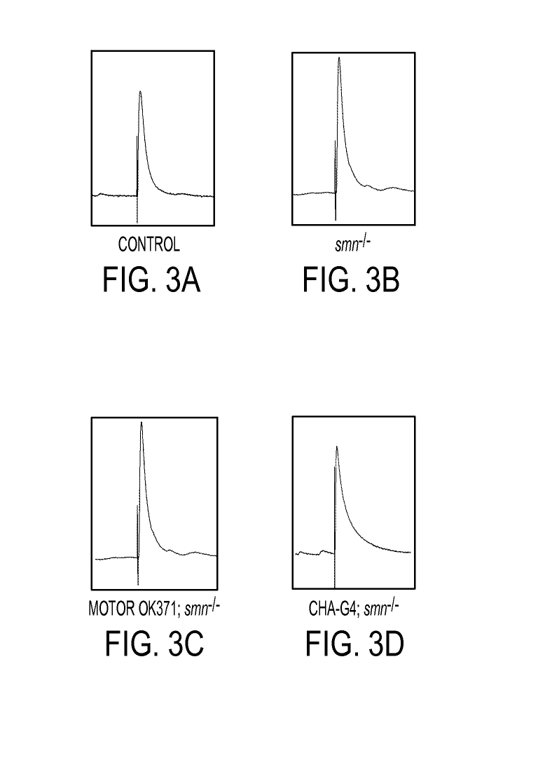

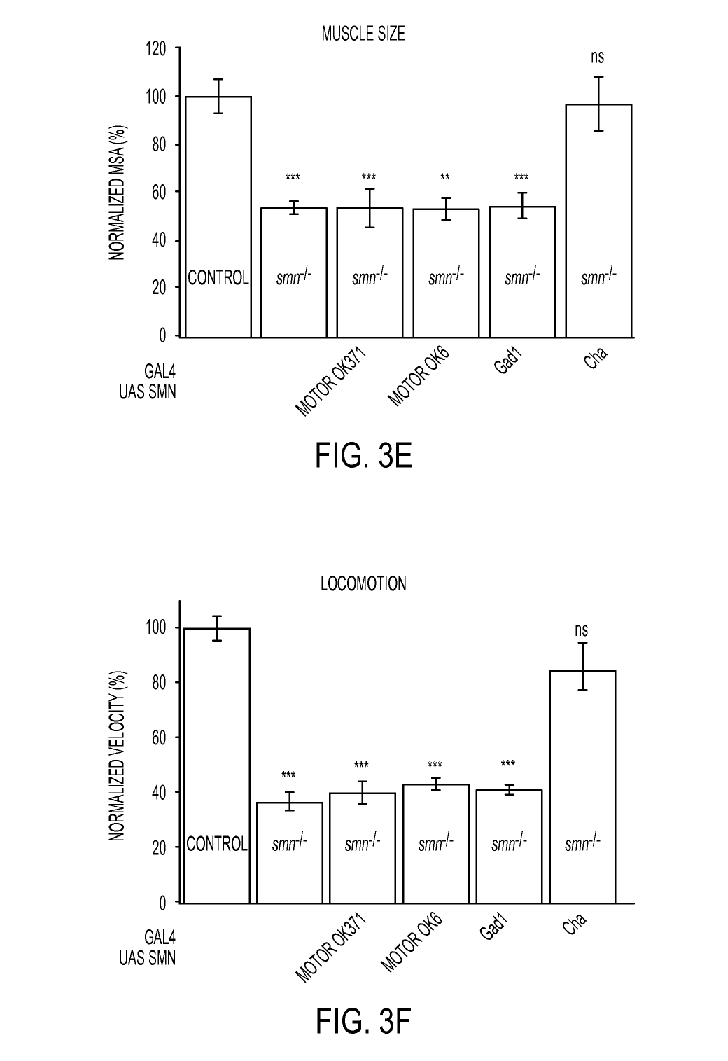

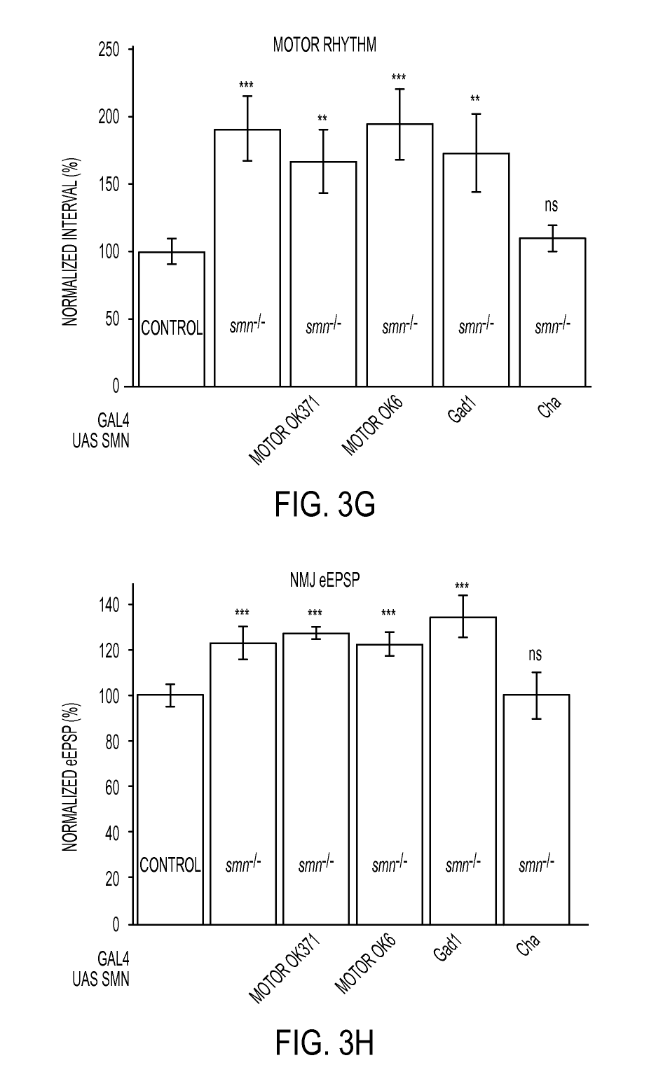

The results in Example 3 show that SMN is required in cholinergic neurons and not in glutaminergic motor neurons, and that SMN is required in both proprioceptive and central cholinergic neurons. Expression of transgenic SMN levels in central cholinergic neurons completely rescued the muscle growth, locomotion and rhythmic activity defects of smn mutants (FIG. 3E-G). Moreover, expression of SMN in cholinergic neurons also fully rescued eEPSP amplitudes at the NMJ terminals of smn mutants to control levels (FIG. 3D, H). Thus, expression of SMN only in cholinergic neurons is sufficient to fully rescue smn mutant phenotypes and can nonautonomously rescue the SMN-dependent defects of both motor neurons and muscles. Further experiments also showed that restoration SMN expression after embryogenesis can rescue smn mutants indicating they do not have persistent defects of motor circuit assembly. There was a differential phenotypic sensitivity to the timing of SMN restoration such that NMJ neurotransmitter was fully corrected by elevating SMN levels at even late stages, while locomotion, motor rhythm and muscle growth required an earlier and longer duration of exposure to increased SMN levels. Finally, inhibition of cholinergic neuron activity replicated a number of the features of smn mutants including non-cell autonomous effects on the neurotransmitter release properties of motor neurons, consistent with cholinergic sensory neurons in the motor circuit having reduced function in smn mutants.

Example 4 Building upon the hypothesis that motor circuits have functional deficits in smn mutants, experiments were designed to test whether increasing the excitability of central cholinergic neurons in these animals could increase motor network activity and alter smn mutant phenotypes. The results of various experiments showed that pharmacological inhibition of K+ channels (using the FDA-approved broad-spectrum K+ channel antagonist 4-AP) positively benefitted smn mutant phenotypes, a result that is consistent with the defective excitability of motor circuits by their interneuron or sensory neuron inputs being a critical consequence of SMN depletion.

### Discussion of Results

The results presented here establish that restoration of SMN in at least two groups of motor circuit neurons (bd and type I md sensory neurons) results in a full rescue of larval phenotypes. The bd and type I md sensory neurons are essential components of a proprioceptive sensory feedback circuit necessary for coordinated contractile locomotion of Drosophila larvae (Hughes and Thomas, 2007). Both the bd and type I md subsets of sensory neurons express the mechanosensitive NompC mechanosensitive NompC TRP channel that is essential for proprioception (Cheng et al., 2010). Sensory feedback does not seem to be necessary for Drosophila larval central pattern generator assembly or basic embryonic and larval movement (Crisp et al., 2008), however without sensory input, both rhythmic motor circuit activity (Fox et al., 2006) and coordinated locomotion behavior is severely disrupted (Hughes and Thomas, 2007; Song et al., 2007). Rescue of SMN in bd and type I md sensory neurons restored the rhythmic motor output of smn mutants, consistent with an important role for sensory input in regulating this activity (Fox et al., 2006). However, restoration of SMN in proprioceptive neurons alone was not sufficient to correct the locomotion velocity of smn mutants indicating that additional neurons require wild-type levels of SMN in order to restore full mobility.

It was discovered that SMN expression in all cholinergic central sensory neurons completely rescued all smn mutant larval phenotypes including locomotion. These results therefore implicate an additional cell autonomous requirement for SMN in one or more groups of central cholinergic neurons. Without being bound by theory, it is possible that these neurons could be descending inputs from the brain (Cattaert and Birman, 2001) or other connections between segmental central pattern generators that promote the coordination necessary for effective locomotion. However, while rescue analysis demonstrated that individual components of the motor circuit can make significant contributions to some smn mutant phenotypes, other phenotypes such as muscle growth additively require SMN expression at normal levels in both central and peripheral cholinergic neurons.

It is curious that only cholinergic motor circuit neurons are selectively susceptible to SMN depletion. In (Lotti, Imlach et al) SMN-dependent defective splicing of a gene required for cholinergic neuron function was identified and it was shown that, like SMN, it must be restored specifically in cholinergic neurons to rescue smn mutant phenotypes. Coupled with the results presented here, it is seen that SMN depletion disrupts the expression of subset of genes, some of which are critically required for the normal function of cholinergic motor circuit neurons. These results establish a mechanistic link between the role of SMN in RNA splicing and the vulnerability of motor circuit function to reduction of SMN.

The basic elements of motor circuits—proprioceptive neurons, interneurons and motor neurons are conserved between Drosophila and humans, even though the neurotransmitters employed in each system are different (Marder and Rehm, 2005). For example, human and mouse motor neurons are cholinergic while proprioceptive neurons are glutamatergic, the inverse of the neurotransmitters employed in Drosophila motor circuits. However, the Drosophila model is nonetheless relevant to treatment of human SMA because prolonging neurotransmitter release by contacting the central sensory neurons with potassium channel activators is not neurotransmitter-specific; the drugs nonspecifically prolong action potentials thereby increasing neurotransmitter release. Without being bound by theory it is possible that cholinergic neurons have a particular and conserved sensitivity to the reduced levels of SMN.

Restoration of SMN in the proprioceptive neurons of Drosophila smn mutants was sufficient to restore normal NMJ neurotransmitter release properties in motor neurons. This suggests that even without direct synaptic contact, increasing SMN in these neurons can influence motor neuron electrophysiological properties, presumably through intermediate interneuron connections. Therefore, it is possible that while the specific details of motor circuit wiring differ between Drosophila and vertebrates, the essential relationships and function of motor networks are conserved and selectively susceptible to depletion of SMN.

Treatment with the small molecule K+ channel antagonist 4-AP and also 4-(dimethylamino) pyridine (data not shown) rescued the Drosophila smn mutant phenotypes. In wild-type animals, 4-AP treatment did not affect muscle size but did reduce locomotion and inhibited NMJ neurotransmitter release as might be anticipated by systemic inhibition of K+channels, which are present throughout the nervous system and in muscles (Wicher et al., 2001). Nonetheless, administration of 4-AP significantly increased both the muscle area and locomotion of smn mutants and fully corrected defects in rhythmic motor output and NMJ neurotransmission.

Treatment with 4-AP has been linked to improvement in function in patients with spinal cord injury, myasthenia gravis and Lambert-Eaton syndrome (Hayes, 2007) and can improve muscle twitch tension in a canine hereditary motor neuron disease (Pinter et al., 1997). A sustained release preparation of 4-AP was recently approved by the FDA for human clinical use in multiple sclerosis (Chwieduk and Keating, 2010). However, until the present discovery, it was not known that SMA involved central sensory neurons at any level.

The data shows that the efficacy of 4-AP in the Drosophila smn mutant model is likely via its activity upon cholinergic neurotransmission in the sensory-motor circuit. Extrapolating this finding to humans, other compounds like 4-AP that can act within the spinal cord to increase neurotransmitter release from sensory neurons, thereby increasing the excitability of motor neural networks and these can also be used as therapeutic agents to ameliorate the symptoms of Spinal Muscular Atrophy.

### EMBODIMENTS OF THE INVENTION

It has now been discovered that SMA can be treated by administering therapeutically effective amounts of 4-AP (or biologically active derivatives or variants thereof) formulated to cross the blood brain barrier. Other potassium channel antagonists can be used in the present invention, including 4-(dimethylamino)pyridine, 4-(methylamino)pyridine and 4-(aminomethyl)pyridine, to treat SMA, either alone or in combination with one another. The therapeutic agents can be administered on the same day or on different days, as discussed below in “pharmaceutical formulations.”

Other K+ channel antagonists for use in the present invention include Dofetilide, Sotalol, Ibutilide (which is approved by the Food and Drug Administration for acute conversion of atrial fibrillation to sinus rhythm), Azimilide, Bretylium, Clofilium, E-4031, Nifekalant, Tedisamil, and Sematilide.

Certain other embodiments are directed to formulations of more than one therapeutic agent, including formulations that optimize the ability of the drugs to cross the BBB.

### Therapeutic K Channel Antagonists and Dosage

4-Aminopyridine is also known as INN fampridine and dalfampridine (Acorda Therapeutics, Inc., New York, marketed under the name Ampyra®). 4-AP is an organic compound with the chemical formula C₅H₄N—NH₂. The molecule is one of the three isomeric amines of pyridine. 4-AP is a relatively selective blocker of members of Kv1 (Shaker, KCNA) family of voltage-activated K+ channels. At concentration of 1 mM it selectively and reversibly inhibits Shaker channels without significant effect on other sodium, calcium, and potassium conductances. While it has long been used as a research tool, in characterizing subtypes of potassium channels, it has now been approved by the FDA to manage some of the symptoms of multiple sclerosis and is indicated for symptomatic improvement of walking in adults with several variations of the disease, (Solari A, Uitdehaag B, Giuliani G, Pucci E, Taus C (2001). Solari, Alessandra. ed. “Aminopyridines for symptomatic treatment in multiple sclerosis”. Cochrane Database Syst Rev (4); Korenke A R, Rivey M P, Allington D R (October 2008). “Sustained-release fampridine for symptomatic treatment of multiple sclerosis”, Ann Pharmacother 42 (10): 1458-65; New Drugs: Fampridine”. Australian Prescriber (34): 119-123, August 2011. The drug has orphan drug status in the United States under the trade name Neurelan. Fampridine is also marketed as Ampyra® in the United States by Acorda Therapeutics (FDA Approves Ampyra to Improve Walking in Adults with Multiple Sclerosis,

Fampridine (4-AP) has also been used clinically in patients with spinal cord injury, myasthenia gravis and Lambert-Eaton syndrome (Hayes, 2007), and can improve muscle twitch tension in a canine hereditary motor neuron disease (Pinter et al., 1997). It is a broad based potassium channel antagonist that prolongs action potentials and consequently increases neurotransmitter release from neurons. The drug has also been shown to reverse tetrodotoxin toxicity in animal experiments. MS patients treated with 4-AP exhibited a response rate of 29.5% to 80%. A long-term study (32 months) indicated that 80-90% of patients who initially responded to 4-AP exhibited long-term benefits. Treatment with 4-AP has been linked to improvement in function in patients with spinal cord injury, myasthenia gravis and Lambert-Eaton syndrome (Hayes, 2007) and can improve muscle twitch tension in a canine hereditary motor neuron disease (Pinter et al., 1997).

Various doses of 4-AP have been tested for clinical use in 17 temperature-sensitive MS patients, with doses ranging from 7.5 to 52.5 mg 4-AP in one to three daily doses at 3- to 4-hour intervals over 1 to 5 days. Thirteen of the 17 patients (76%) given 4-AP showed clinically important motor and visual improvements compared with the placebo group. Seventy percent of the daily 4-AP improvements lasted 7 to 10 hours. The improvements for two consecutive doses of 4-AP lasted a mean of 7.07 hours (83% of the average 8.53-hour treatment-observation period) compared with 2.36 hours for placebo (26% of the average 9.06-hour treatment-observation period). No serious side effects occurred. Stefoski D, et al.; Neurology. 1991 September; 41(9):1344-8; 4-Aminopyridine in multiple sclerosis: prolonged administration. The FDA-approved dose 10 mg administered orally 2 times per day.

Therapeutically effective amounts will vary depending on various factors including: 1. whether one or more than one agents is administered; 2. the efficacy of the agent, alone or combined with other agents; 3. the type of formulation, such as a sustained-release formulation that may have higher amounts since the drug is released slowly; 4. the age of the patient, severity of the disease; 5. the frequency of administration; and 6. the individual subject's tolerance of and response to the agent.

### Pharmaceutical Formulations and Administration

In certain embodiments, multiple therapeutically effective doses of one or more the therapeutic agents are administered in a single day, or over the course of weeks or months, as needed to ameliorate one or more symptoms of the disease. The therapeutic agents can be administered individually (i.e. treatment with only one specific agent), or more than one agent can be administered either separately or in combination. The agents can be administered once or more than once per day. The therapeutically effective amounts of the different agents may vary depending on the specific agent and whether the agent is administered alone or together with another agent. The therapeutically effective amount will also vary based on the particular formulation.

Pharmaceutical compositions for use in the present methods include therapeutically effective amounts of one or more of the therapeutic agents, i.e., an amount sufficient to prevent or treat the diseases described herein in a subject, formulated for local or systemic administration. The subject is preferably a human but can be non-human as well. A suitable subject can be an individual who is suspected of having, has been diagnosed as having, or is at risk of developing one of the described diseases.

Active agents for therapeutic administration are preferably low in toxicity and cross the blood brain barrier. The progress of this therapy is easily monitored by conventional techniques and assays that may be used to adjust dosage to achieve a desired therapeutic effect.

A composition of the therapeutic agents can also include a pharmaceutically acceptable carrier. As used herein the language “pharmaceutically acceptable carrier” includes solvents, dispersion media, coatings, antiviral agents, antibacterial agents, antifungal agents, isotonic and absorption delaying agents, and the like, compatible with pharmaceutical administration. Supplementary active compounds can also be incorporated into the compositions. Other topical formulations are described in Sheele et al., 7, 151, 091.

Therapeutic compositions may contain, for example, such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70% active ingredient.

The therapeutic agents can also be mixed with diluents or excipients which are compatible and physiologically tolerable as selected in accordance with the route of administration and standard pharmaceutical practice. Suitable diluents and excipients are, for example, water, saline, dextrose, glycerol, or the like, and combinations thereof. In addition, if desired, the compositions may contain minor amounts of auxiliary substances such as wetting or emulsifying agents, stabilizing or pH buffering agents.

In some embodiments, the therapeutic compositions of the present invention are prepared either as liquid solutions or suspensions, or in solid forms, preferably for oral administration. The formulations may include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. Solutions, suspensions, or sustained release formulations typically contain 1%-95% of active ingredient, preferably 2%-70%.

The formulations may also contain more than one therapeutic agent as necessary for the particular indication being treated, preferably those with complementary activities that do not adversely affect each other. Such molecules are suitably present in combination in amounts that are effective for the purpose intended.

Suitable examples of sustained release preparations include semipermeable matrices of solid hydrophobic polymers containing the therapeutic agents, which matrices are in the form of shaped articles, e.g., films, or microcapsule. Examples of sustained release matrices include, but are not limited to, polyesters, hydro gels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPOT (injectable micro spheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The therapeutic agents of the present invention may be formulated for administration by any suitable means as long as they cross the blood brain barrier (BBB). Strategies for formulating therapeutic agents that cross the BBB are well known and include the following:

Increasing the permeability (opening) of the BBB
Osmotic opening of the blood-brain barrier
Chemical opening
Cerebral vasodilatation: stimulation of the sphenopalatine ganglion, nitric oxide inhalation
Disruption of blood-brain barrier by focused ultrasound

Pharmacological strategies to facilitate transport across the BBB
Modification of the drug to enhance its lipid solubility
Use of transport/carrier systems
Inhibition of efflux transporters that impede drug delivery
Trojan horse approach
Chimeric peptides
Monoclonal antibody fusion proteins
Prodrug bioconversion strategies
Nanoparticle-based technologies
Neuroimmunophilins

An alternative for delivering drugs to the brain is direct administration to the brain or spinal cord, thus bypassing the BBB. This can be accomplished for example by injection into epidural venous plexus or direct introduction of drugs into the brain, spinal column or the cerebrospinal fluid (CSF). Drug pumps could facilitation continual administration of the therapeutic agents in the methods of the present invention.

Liposomes can be useful in delivery of small molecules to the brain. Particularly useful liposomes can be generated by, for example, the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Polypeptides of the present invention can be conjugated to the liposomes as described in, for example, Werle et al., Int. J. Pharm. 370(1-2): 26-32 (2009).

For a review of methods for formulating drugs for delivery to the brain or spinal cord see Reinhard Gabathuler, Neurobiology of Disease 37 (2010) 48-57Approaches to transport therapeutic drugs across the blood-brain barrier to treat brain diseases. See also Journal of Drug Delivery Volume 2011, Article ID 469679, doi:10.1155/2011/469679. Review Article Carlos Spuch and Carmen Navarro, Liposomes for Targeted Delivery of Active Agents against Neurodegenerative Diseases (Alzheimer's Disease and Parkinson's Disease).

For in vivo administration, the pharmaceutical compositions are preferably administered orally or parenterally, i.e., intraarticularly, intravenously, intraperitoneally, subcutaneously, or intramuscularly. In particular embodiments, the pharmaceutical compositions are administered intravenously or intraperitoneally by a bolus injection. Stadler, et al., U.S. Pat. No. 5,286,634. For the prevention or treatment of disease, the appropriate dosage will depend on the severity of the disease, whether the drug is administered for protective or therapeutic purposes, previous therapy, the patient's clinical history and response to the drugs and the discretion of the attending physician.

The resulting pharmaceutical preparations may be sterilized by conventional, well known sterilization techniques. The aqueous solutions can then be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the lipidic suspension may include lipid-protective agents which protect lipids against free-radical and lipid-peroxidative damages on storage. Lipophilic free-radical quenchers, such as a-tocopherol and water-soluble iron-specific chelators, such as ferrioxamine, are suitable.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application.

The pharmaceutical compositions of this invention may, for example, be placed into sterile, isotonic formulations with or without cofactors which stimulate uptake or stability. The formulation is preferably liquid, or may be lyophilized powder. For example, the compositions of the invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

Suitable Solvates Include Hydrates. Suitable salts include those formed with both organic and inorganic acids or bases. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Formulations of use in the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described.

Therapeutic agents of the present invention may be administered simultaneously meaning the administration of medicaments such that the individual medicaments are present within a subject at the same time. In addition to the concomitant administration of medicaments (via the same or alternative routes), simultaneous administration may include the administration of the medicaments (via the same or an alternative route) at different times.

### DEFINITIONS

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane Antibodies: A Laboratory Manual Cold Spring Harbor Laboratory Press, Cold Spring Harbor, N.Y. (1990); Principles of Neural Science, 4^{th} ed., Eric R. Kandel, James H. Schwart, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The terms “individual,” “subject,” and “patient” are used interchangeably herein and refer to any mammalian subject for whom diagnosis, treatment, or therapy is desired, particularly humans. A “subject” as used herein generally refers to any living multicellular organism. Subjects include, but are not limited to animals (e.g., cows, pigs, horses, sheep, dogs and cats) and plants, including hominoids (e.g., humans, chimpanzees, and monkeys). The term includes transgenic and cloned species. The term “patient” refers to both human and veterinary subjects.

“Administering” shall mean delivering in a manner which is affected or performed using any of the various methods and delivery systems known to those skilled in the art. Administering can be performed, for example, orally, or intravenously, via implant, transmucosally, transdermally, intradermally, intramuscularly, subcutaneously, or intraperitoneally. Administering can also be performed, for example, once, a plurality of times, and/or over one or more extended periods.

The phrase “therapeutically effective amount” means an amount sufficient to produce a therapeutic result. Generally the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition. For example, eliminating or reducing or mitigating the severity of a disease or set of one or more symptoms. The full therapeutic effect does not necessarily occur by administration of one dose and may occur only after administration of a series of doses. Thus, a therapeutically effective amount may be administered in one or more administrations.

“Treating” a disease means taking steps to obtain beneficial or desired results, including clinical results, such as mitigating, alleviating or ameliorating one or more symptoms of a disease; diminishing the extent of disease; delaying or slowing disease progression; ameliorating and palliating or stabilizing a metric (statistic) of disease. “Treatment” refers to the steps taken.

“Mitigating” means reducing or ameliorating a disease or symptom of a disease. For example, mitigation can be achieved by administering a therapeutic agent before the phenotypic expression of the disease (i.e. prior to the appearance of symptoms of the disease) Mitigation includes making the effects of disease less severe by avoiding, containing, reducing or removing it or a symptom of it. Mitigating an enumerated disease as described herein comes within the definition of “treating” an enumerated disease before symptoms occur. Amounts of therapeutic agents that mitigate a disease are herein referred to as “therapeutically effective amounts.”

### EXAMPLES

### Materials and Methods

Drosophila stocks. smnX7 (Chang et al., 2008) is small deletion that removes the entire smn coding region from 93 bp upstream of the transcription start site up to the last 44 bp of the 3′ UTR without disrupting other loci. Under conventional culture conditions, the number of homozygous smnX7 mutants that survive to the third instar is few consistent with previous reports (Chang et al., 2008), however if stocks were cultured at low density, an increase the numbers that survived to this stage could reliably be identified. To do this, parental stocks and controls were allowed to lay overnight on grape juice agar plates supplemented with yeast paste, the plates were then removed and cultured at 25° C. until animals reached the third larval instar. smn73Ao is a strong loss-of-function point mutant allele that produces an unstable protein. The results differ from previous electrophysiological observations based on the SMN73Ao allele which reported a decrease in evoked neurotransmitter release at the NMJ of these animals (Chan et al., 2003). This finding was replicated in one SMN73Ao stock (B#4802), however, this defect could not be rescued by transgenic SMN (data not shown). A different SMN73Ao stock (Gift from Greg Matera, UNC), confirmed to have low SMN, and which had gone through multiple backcrosses to a wild-type background had an similar increase in eEPSP amplitude to smnX7 and other smn mutant alleles both in homozygous and in transheterozygous combinations (Supplementary FIG. 2D). It was concluded that the previous finding may have been caused by a second site mutation. Similarly, x no change in morphological synaptic boutons was observed in smnX7 mutants even though a decrease had previously been reported for the SMN73Ao allele (Chan et al., 2003; Chang et al., 2008). These results are consistent with observations of other ‘strong’ smn alleles which also show little or no NMJ morphological change (Chang et al., 2008). As in previous studies, ubiquitous expression of transgenic SMN can rescue both adult viability, locomotion and muscle size (Chan et al., 2003; Chang et al., 2008; Rajendra et al., 2007), however it was discovered that only neuronal only expression of SMN with either nsyb-Gal4 or geneswitch elav-Gal4 is required to rescue locomotion in SMN mutants in contrast to previous reports that muscle or neuron expression was sufficient (Chan et al., 2003). One possible explanation for this discrepancy is that the mesodermal how24B-Gal4 driver (Brand and Perrimon, 1993) used in these studies has significant neuronal expression in addition to strong muscle expression. UAS::Flag-Smn—full length Smn protein with amino-terminal Flag sequence inserted on chromosome II (Chang et al., 2008). Targeted expression was confirmed using anti-Flag immunohistochemistry (data not shown). UAS-PLTXII (Membrane tethered PLTXII assembled from N to C terminus as a secretory signal sequence, the mature cleaved PLTX-II peptide sequence, a hydrophilic linker sequence with an embedded c-Myc epitope tag, and a GPI targeting sequence (B. C, Michael Nitabach and B D M unpublished) Gal4 lines: nsyb-Gal4 (Bushey et al., 2009), OK6-Gal4, G14-Gal4 (Aberle et al., 2002), OK371-Gal4 (Mahr and Aberle, 2006), Actin-Gal4 (Ito et al., 1997), Cha-Gal4 (Salvaterra and Kitamoto, 2001), clh201-Gal4, 1003.3-Gal4 (Hughes and Thomas, 2007), ppk-Gal4 (Ainsley et al., 2003) NP2225-Gal4 (Sugimura et al., 2003), Gad1-Gal4 (Ng et al., 2002) and elav-geneswitch (Osterwalder et al., 2001). UAS lines: UAS-Flag-Smn (Chang et al., 2008), UAS-Kir2.1 (Paradis et al., 2001), UAS-PLTXII and UAS-SDN (Mosca et al., 2005) Electrophysiology. NMJ electrophysiology: Intracellular recordings from muscle 6, segment A3 were performed as previously described (Imlach and McCabe, 2009). Briefly, third instar larvae were dissected and recordings carried out in HL3 saline containing 1.0 mM Ca2+. Data was only analyzed from recordings where the resting membrane potential was less than −55 mV. Recordings were performed using an Axoclamp 2B amplifier. Data were low-pass filtered at 1 kHz, digitized, and recorded to disk using a Digidata 1322A interface. Both eEPSPs and mEPSPs amplitudes were measured using the peak detection feature of MiniAnalysis program (Synaptosoft, Inc.). All events were verified manually while blinded to genotype. The amplitude and the frequency of mEPSPs were calculated from continuous recordings in the absence of stimulation (50-100 s). Quantal content was calculated for each individual recording by calculating (eEPSP amplitude/mEPSP amplitude) (Davis et al., 1998) after correction for nonlinear summation errors (Martin, 1955). Motor rhythm: Spontaneous motor rhythm was recorded as previously described (Fox et al., 2006). Briefly, recordings were made from muscle 6 of abdominal segment A1 in standard saline (Jan and Jan, 1976) where CNS and motor neurons were intact. To measure the average inter event interval, the peak detection feature of MiniAnalysis (Synaptosoft, Inc.) was used detect all spontaneous eEPSPs events that occurred over a 3 minute period. Locomotion: Assays were essentially performed as previously described (Suster and Bate, 2002). Briefly, single larva were placed on 1% agarose plates on a lightbox at 25° C. and 70% humidity. After 1 minute to acclimatize, video recordings of the locomotion path were captured with camera Sentech STC-620CC video camera mounted on a EMZ-8TR microscope and recorded with Final Cut Express v 4.0 (Apple). These were converted to quicktime .mov files with QuickTime v7.6.4 (Apple). Path length and velocity was then analyzed using DIAS v 3.4.2 (Soll Technologies) software. NMJ immunohistochemistry. Wandering third instar larvae were dissected and stained as previously described (Brent et al., 2009a; Brent et al., 2009b). Type Ib and Is boutons were counted using a 40× objective on a Zeiss Axio Imager.Z1 microscope at muscle 4 abdominal segment A3. Antibodies used were mouse anti-cysteine string protein (CSP, 1:200, Developmental Studies Hybridoma Bank at the University of Iowa), Cy5-conjugated goat anti-horseradish peroxidase (1:400, Jackson ImmunoResearch) and Goat anti-mouse Alexa488 (1:2000, Invitrogen). Larval preparations were imaged on a Zeiss LSM 510 Confocal microscope.

Western blotting. The following monoclonal antibodies were used for western blotting: Drosophila-specific anti-SMN (Chang et al., 2008), anti-B actin (Sigma), anti-Tubulin DM 1A (Sigma) and anti-FLAG (Sigma). Total protein extracts for Western blot analysis were prepared by homogenization of Drosophila third-instar larvae in SDS sample buffer (2% SDS, 10% glycerol, 5% bmercaptoethanol, 60 mM Tris-HCl pH 6.8, bromophenol blue) followed by brief sonication and boiling. Protein concentration was measured using RC DC Protein Assay (Bio-Rad). All protein samples were analyzed by SDS/PAGE on 12% polyacrylamide gels followed by transfer onto nitrocellulose membrane.

Drosophila stocks: smnX7 (Chang et al., 2008), smn73Ao (Chan et al., 2003), smnE33 (Rajendra et al., 2007).

Muscle measurement: Muscle area measurements were carried out at muscle 6 of segment A3 of phallodin stained muscle fillet preparations (Brent et al., 2009).

Locomotion: Larval locomotion assays were essentially performed as previously described (Suster and Bate, 2002) (see supplemental information).

Motor rhythm: Spontaneous motor rhythm was recorded as previously described (Fox et al., 2006). To measure the average inter-spike interval, the peak detection feature of MiniAnalysis (Synaptosoft, Inc.) was used detect all spontaneous eEPSPs events that occurred over a 3 minute period.

NMJ electrophysiology: Intracellular recordings from muscle 6, segment A3 were performed as previously described (Imlach and McCabe, 2009).

Drug treatment: Gene-switch GAL4 SMN expression was induced by culturing larvae with RU486 at 10 g/ml for 148 hours, 96 hours, 72 hours or 48 hours prior to phenotypic measurement in SMN mutants (controls were all assayed at wandering L3 stage). SMN induction was confirmed by western blot. For 4-AP treatment, 2 mM 4-AP (Sigma) was added to the yeast paste on which larvae were cultured immediately after hatching and throughout the subsequent larval period.

Statistical methods: Significance was tested by ANOVA as indicated using Instat 3.0 (GraphPad). In all FIGS., error bars represent the standard error of the mean and *=p<0.05, **=p<0.01, ***=p<0.001.

### Example 1

### Validation of the Drosophila Smn Mutant Model

To model the low level of SMN found in SMA patients in Drosophila, the zygotic protein null SMN allele (smnX7) was used, which has a small deficiency that removes the entire smn coding region without disrupting nearby loci (Chang et al., 2008). The remaining SMN in these animals is contributed by maternal protein which provided <6% the level of SMN compared to controls at the third instar larval stage (Supplementary FIG. 1A). smnX7 mutants never initiated pupation but instead persisted as third instar larvae, often surviving over 5 days at this stage, consistent with previous observations of other smn mutant alleles (Chan et al., 2003).

To confirm this phenotype was dependent on SMN, a transgenic UAS flag-tagged SMN construct was ubiquitously expressed (Chang et al., 2008) in smnX7 mutants using Actin-Gal4. This restored normal pupation of smnX7 mutants with 100% of larvae initiating pupation and >80% subsequently closing to produce viable adults (data not shown). Thus, smnX7 mutants have low levels of SMN at late larval stages and can be rescued by transgenic SMN. This mutant allele was used for all subsequent experiments except where noted.

Drosophila smn mutant larvae were smaller than control animals. In order to examine if this was associated with a reduction in muscle size, smn mutant and control larval muscles were labeled with Phalloidin. The results showed that smn mutants had a 46% (P<0.001) reduction in muscle surface area compared to controls (FIG. 1A-C, see also Table Si). This defect was fully rescued by ubiquitous expression of transgenic SMN. Smn mutant larvae were sluggish and moved less frequently than controls. To quantify this defect, video capture and tracing software was used to measure the locomotion of smn mutant and control larvae. Results showed that smn mutants showed a 63% (P<0.001) decrease in locomotion velocity compared to control animals which was restored to control levels by ubiquitous expression of transgenic SMN (FIG. 1D-F). Thus, similar to SMA patients, Drosophila with low levels of SMN have muscle and locomotion defects.

Locomotion of Drosophila larvae has been linked to the rhythmic activity of segmental central pattern-generating networks (CPGs) in the ventral nerve cord (VNC) (Fox et al., 2006) which receive inputs from both the brain hemispheres (Cattaert and Birman, 2001) and proprioceptive sensory neurons (Cheng et al., 2010; Hughes and Thomas, 2007; Song et al., 2007) and output activity to motor neurons. To measure the activity of these pattern-generating neurons, the spontaneous activity of motor neurons was recorded in preparations where the left the brain and VNC was left in situ (Fox et al., 2006). In control animals, periodic bursting of motor activity at regular intervals was consistent with previous studies (Cattaert and Birman, 2001; Fox et al., 2006) (FIG. 1G). In contrast, this activity was disrupted in smn mutants which had short irregular bursts that varied in duration (FIG. 1H). This defect was quantified by measuring the average inter-spike interval between all spontaneous spike events in smn mutants and controls over a fixed time period. Compared to controls, smn mutants showed a 90% (P<0.001) increase of inter-spike interval (FIG. 1I). As with locomotion, normal rhythmic motor activity was fully restored by ubiquitous expression of transgenic SMN. Thus, the output of motor circuits is defective in Drosophila smn mutants.

### Neurotransmitter Release Properties of Individual Motor Neurons

The brain was removed and motor neurons were stimulated directly using a suction electrode (Imlach and McCabe, 2009). Compared to controls, there was a 23% (P<0.005) increase of evoked Excitatory Post-Synaptic Potential (eEPSP) amplitude at the NMJs of smn mutants (FIG. 1J-L). The increase of NMJ eEPSP amplitude in smnX7 mutants was restored to control levels by ubiquitous expression of transgenic SMN (FIG. 1L). A60% (P<0.05) increase in miniature Excitatory Post-Synaptic Potential (mEPSP) frequency was also observed. In contrast mEPSP amplitude at smn mutant NMJ terminals was not different to controls (Supplementary FIG. 2A,B) leading to a 64% (P<0.001) increase in quantal content (Supplementary FIG. 2C). These findings are consistent with a presynaptic change in the neurotransmitter release properties of motor neurons in smn mutants. Trans-heterozygous combinations of smnX7 with other smn mutant alleles was done and the results confirmed similar changes in eEPSP amplitudes in these mutants that were not found in heterozygous smnX7 animals (Supplementary FIG. 2D). When the morphological features of smnX7 mutant NMJs was studied there was no significant difference in the number of synaptic boutons compared to controls (Supplementary FIG. 2E). In summary, Drosophila smn mutants have increased NMJ evoked neurotransmitter release that is accompanied by defects of muscle growth, locomotion and motor rhythm.

### Example 2

### Restoration of SMN in the Nervous System Rescues Smn Mutant Phenotypes

Depletion of SMN perturbed multiple outputs of the Drosophila motor system in addition to muscle growth. In order to identify the cell autonomous requirement for normal SMN levels, iteratively more tissue-restricted Gal4 drivers were used to assess rescue of smn mutants. First, transgenic SMN was expressed only in the muscles of smnX7 mutants using G14-Gal4, a larval muscle-specific driver. This produced no significant increase in muscle surface area (FIG. 2C,E) or effects upon the locomotion, rhythmic motor output and NMJ eEPSP amplitude of smn mutants (FIG. 2F-H).

Next, SMN restoration was tested only in the nervous system of smn mutants using the neuron-specific nsyb-Gal4 driver. In contrast to muscle restoration of SMN, pan-neuronal restoration of SMN fully rescued the muscle surface area of smn mutants to control levels (FIG. 2 B,D,E) and also completely restored their locomotor velocity, rhythmic motor output and NMJ eEPSP amplitudes (FIG. 2F-H). Neuron-only rescue of smn mutants was not sufficient to produce viable Drosophila adults (data not shown), presumably due to the SMN level in tissues that are not rescued becoming fully depleted to the point where cellular viability is compromised (Chan et al., 2003). The results established that the defects of muscle growth in smn mutant larvae are due to a non-autonomous requirement for normal SMN levels in the nervous system rather than in muscle fibers themselves.

### Example 3

### SMN is Required in Cholinergic Neurons and not in Motor Neurons

The Drosophila VNC, like the human spinal cord, is populated by neurons with diverse neurotransmitter expression. All Drosophila motor neurons in addition to a subset of central interneurons are glutamatergic (Daniels et al., 2008). Because there are presynaptic defects in neurotransmitter release at the NMJ in smn mutants, the ability of transgenic SMN expression in motor neurons to rescue smn mutants was tested. OK371-Gal4 was used as an enhancer trap inserted in the vesicular glutamate transporter promoter to express transgenic SMN only in the glutamatergic neurons of smn mutants. This produced no difference in muscle surface area, locomotion velocity or rhythmic motor output compared to smn mutants alone (FIG. 3F-G). Surprisingly, there was no reduction of the aberrant increase of eEPSP amplitude at the NMJs of these animals (FIG. 3B,C,E). This unexpected result was confirmed using a second independent motor neuron-specific driver OK6-Gal4 (FIG. 3E-H). Therefore, similar to the requirement for SMN in muscle growth, the aberrant neurotransmitter release at the NMJ of smn mutants is not the result of the cell autonomous loss of SMN in motor neurons. This result prompted investigation to determine if SMN was required in other neuron types in the Drosophila motor circuit.

Inhibitory inputs are important regulators of motor circuit function (Featherstone et al., 2000) so the glutamic acid decarboxylase 1 promoter Gal4 was used to restore of SMN in gabaergic neurons, however no significant rescue of any smn mutant phenotype was seen (FIG. 3EH). The majority of excitatory neurons in the Drosophila nervous system are cholinergic (Salvaterra and Kitamoto, 2001) and motor neurons receive synaptic input from cholinergic neurons (Baines, 2006). Therefore transgenic SMN was restored in smn mutants using choline acetyltransferase (Cha) promoter-driven Gal4. In contrast to glutamatergic and gabaergic drivers, expression of transgenic SMN levels in cholinergic neurons completely rescued the muscle growth, locomotion and rhythmic activity defects of smn mutants (FIG. 3E-G). Moreover, expression of SMN in cholinergic neurons also fully rescued eEPSP amplitudes at the NMJ terminals of smn mutants to control levels (FIG. 3D,H). Thus, expression of SMN only in cholinergic neurons is sufficient to fully rescue smn mutant phenotypes and can nonautonomously rescue the SMN-dependent defects of both motor neurons and muscles.

### SMN is Required in Both Proprioceptive and Central Cholinergic Neurons

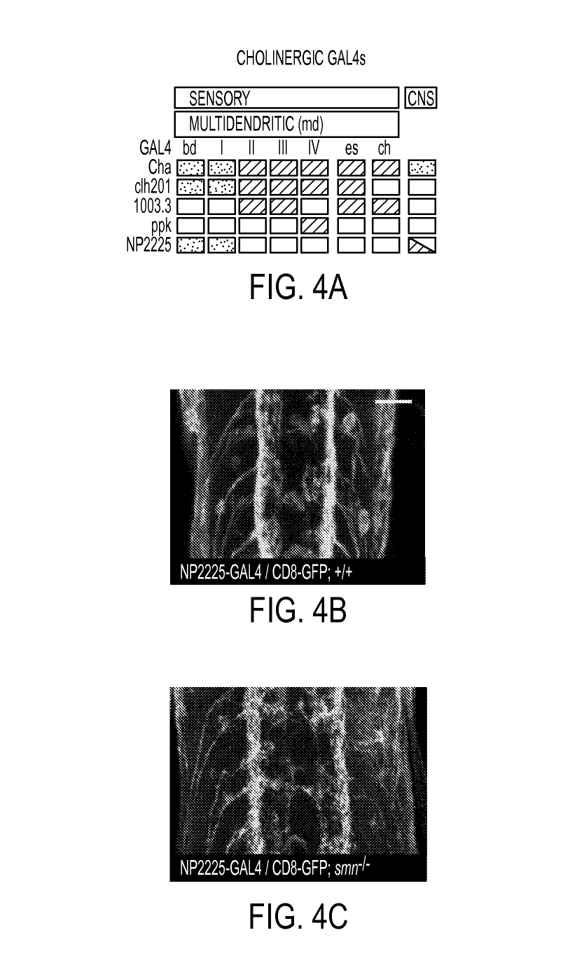

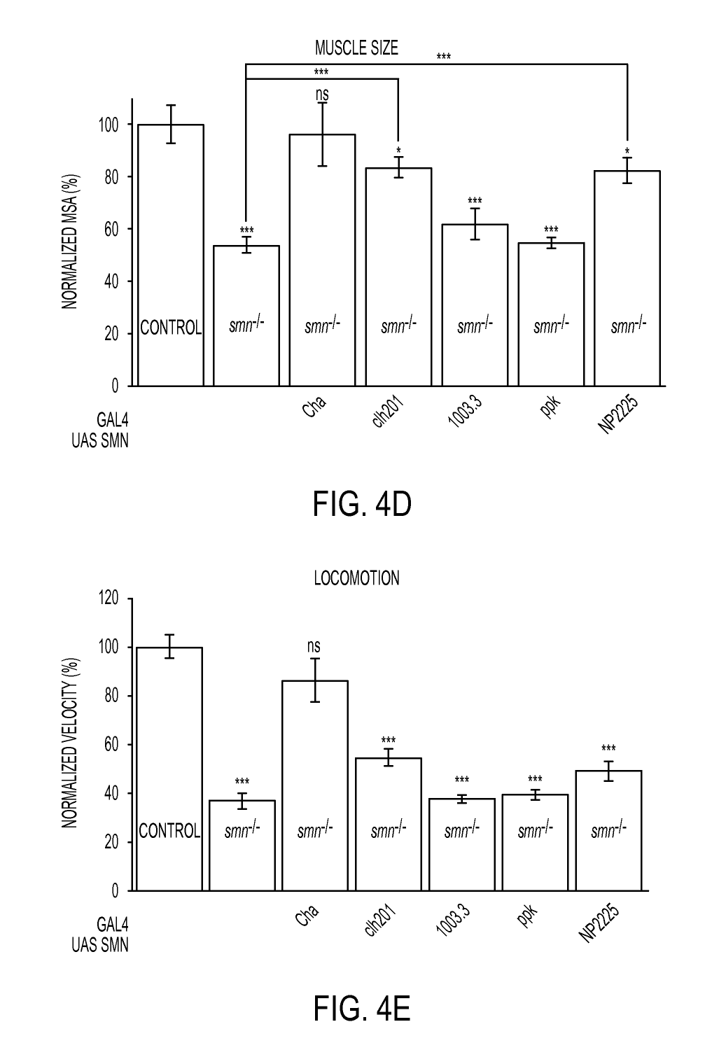

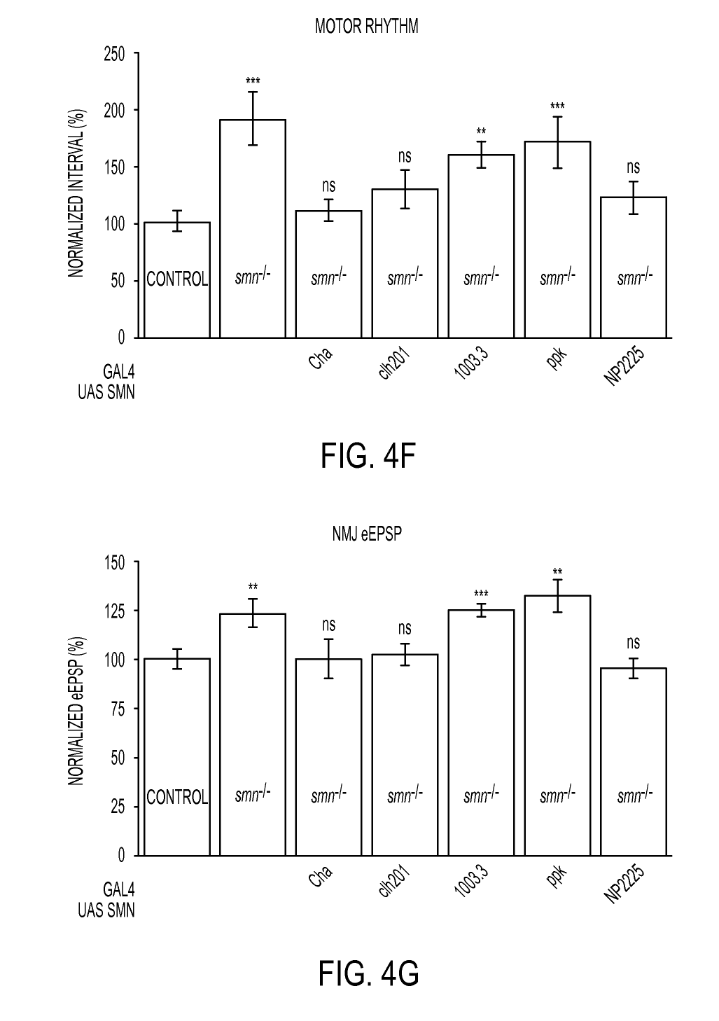

All Drosophila larval sensory neurons in addition to the majority of excitatory central neurons are cholinergic (Salvaterra and Kitamoto, 2001). To dissect the requirement for normal SMN levels between these two populations, the ability of transgenic SMN expression in sensory neurons alone to rescue smn mutant phenotypes was determined. Drosophila sensory neurons are categorized into three major types—multiple dendrite neurons (md) of which there are 5 subclasses (bd, I, II, III and IV), external sense organ neurons (es) and chordotonal neurons (ch). A panel of sensory neuron Gal4 drivers (FIG. 4A) was used to restore SMN only in major types of sensory neurons. When SMN was expressed in all md neurons and es sensory neurons but not ch neurons or central neurons, both the rhythmic motor output and evoked NMJ eEPSP amplitudes of smn mutants were restored to control levels and muscle surface area was increased to 83.5% of controls (P<0.05) (FIG. 4D,F,G). However expression of transgenic SMN with this driver did not significantly change the locomotion of smn mutants (FIG. 4E). In contrast, expression of SMN in ch neurons did not rescue any smn mutant phenotype (FIG. 4D-G). Using additional Gal4 drivers that are expressed in smaller subsets of md or es sensory neurons (FIG. 4A), it was discovered that it was sufficient to restore SMN only in bd and type I md neurons to rescue defects of rhythmic motor output and NMJ neurotransmission and increase the muscle growth of smn mutants (FIG. 4D,F,G). Expression of SMN in both the CNS and peripheral cholinergic neurons with Cha-Gal4 fully rescues all phenotypes including locomotion and muscle size (FIG. 4D-G). This shows that, in addition to bd and type I md sensory neurons, SMN must be restored in at least one other additional population of cholinergic neurons that resides within the CNS to completely correct smn mutant locomotion and fully restore muscle size. Both bd and type I md sensory neurons have recently been demonstrated to be required for propioceptive feedback to the motor circuit of Drosophila larvae (Cheng et al., 2010; Hughes and Thomas, 2007). To determine if these neurons were morphologically disrupted by SMN depletion, the sensory or axonal processes of the bd and type I md neurons labeled in smn mutants were examined, however no obvious defects were found in sensory processes (data not shown) and the axons of these neurons projected into the CNS similarly to controls (FIG. 4 B,C). The data therefore showed that reduced SMN in proprioceptive neurons might disrupt their function rather than their development or connectivity.

Smn Mutant Phenotypes can be Rescued after Embryogenesis

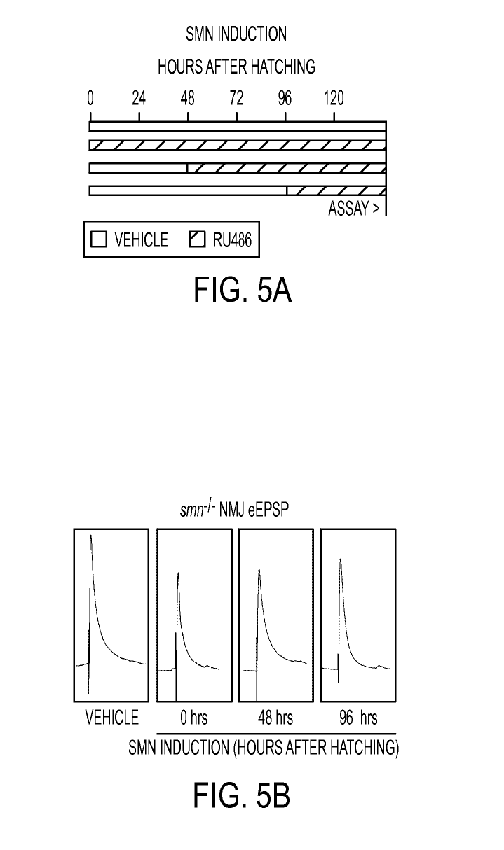

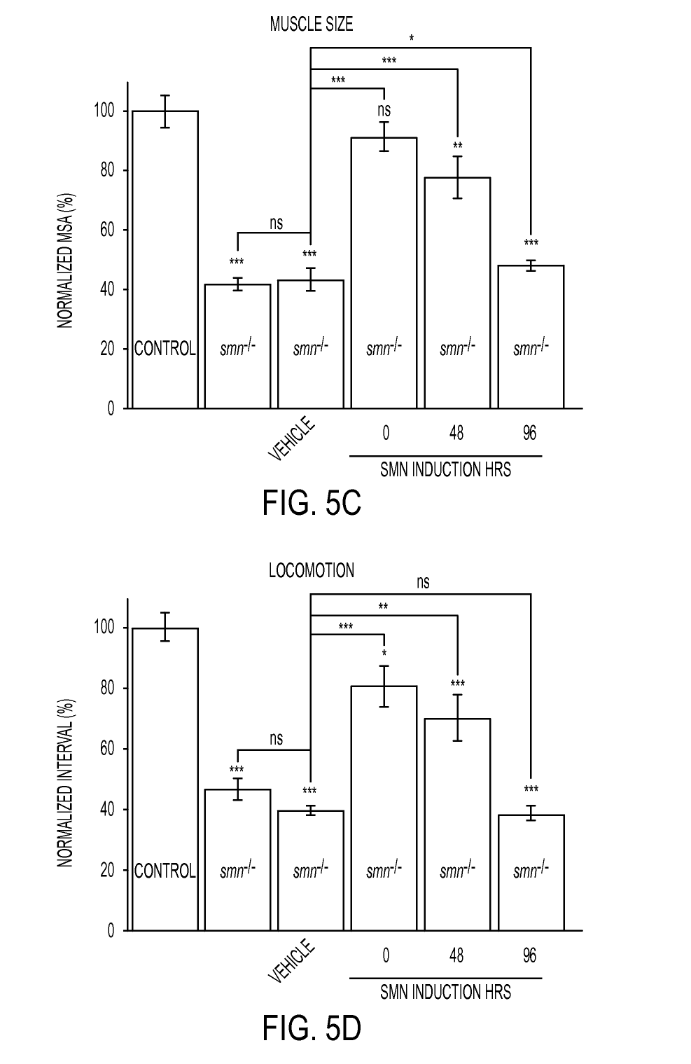

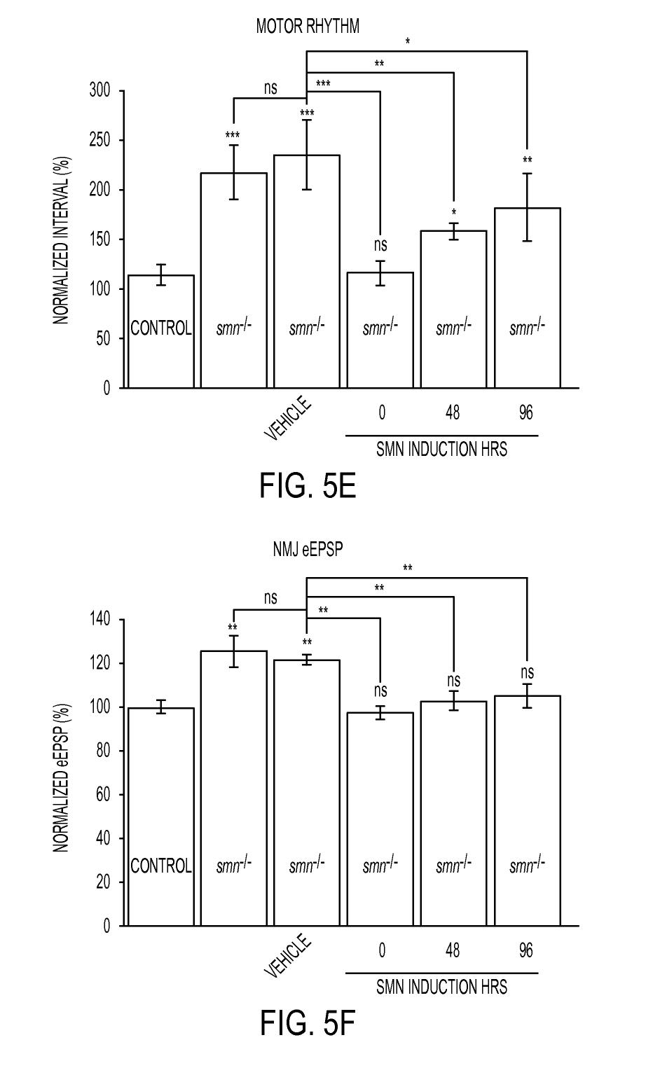

Drosophila larval neurons develop, connect and become functional during the 24 hours of embryonic development prior to hatching (Baines, 2006). To determine if SMN depletion could have disrupted the nervous system assembly during this period the ‘geneswitch’ RU486-drug inducible Gal4 system was used to control the temporal restoration of transgenic SMN. To ask if smn mutant phenotypes could be rescued by expression of transgenic SMN subsequent to the completion of embryogenesis smn mutant larvae and controls carrying the neuron-specific elav-geneswitch driver were exposed to RU486 containing media immediately after hatching and throughout the subsequent larval period (FIG. 5A). When transgenic SMN expression was not induced there was no difference compared to smn mutants alone (FIG. 5C-F). In contrast, when SMN expression was induced immediately after embryogenesis, third instar larval muscle size, locomotion, rhythmic motor output and motor neuron eEPSP amplitudes were indistinguishable from control animals (FIG. 5B-F). This result established that restoration SMN expression after embryogenesis can rescue smn mutants which show they do not have persistent defects of motor circuit assembly.

SMN expression in the nervous system of smn mutants was delayed until progressively later larval stage. When transgenic SMN was induced in smn mutants at 48 or 96 hours after embryo hatching, intermediate phenotypes were found where muscle volume, motor rhythm defects and locomotion where only partially restored compared to controls (FIG. 5C-D). In contrast, NMJ eEPSP amplitudes were completely restored in smn mutants to control levels by only 48 hours of SMN expression (FIG. 5B,F). These results revealed a differential phenotypic sensitivity to the timing of SMN restoration with NMJ neurotransmitter fully corrected by elevating SMN levels at even late stages, while locomotion, motor rhythm and muscle growth required an earlier and longer duration of exposure to increased SMN levels.

### Inhibiting Cholinergic Neuron Activity Mimics Aspects of SMN Depletion.

During Drosophila embryonic development, complete removal of cholinergic input onto motor neurons results in motor neuron hyperexcitability and increased neurotransmission (Baines et al., 2001). It was hypothesized that the non-autonomous changes of motor neuron properties in smn mutants might be explained by defective excitatory input from cholinergic neurons in the motor circuit. Complete loss or inhibition of all cholinergic neuron activity results in embryonic lethality (Kitamoto et al., 2000). To test this hypothesis, transgenic tools designed to partially inhibit neurotransmission in cholinergic neurons were employed. Lines were used that either express moderate levels of the human inward rectifying channel Kir2.1 which inhibits membrane depolarization (Paradis et al., 2001) or express membrane-tethered Plectreurys Toxin II (PLTXII) which inhibits synaptic N-type voltage gated calcium channels (B. C., Michael Nitabach and B. D. M, unpublished). To determine the effectiveness of this approach, these transgenes were first expressed in motor neurons alone using OK6-Gal4. It was discovered that Kir2.1 reduced eEPSP amplitudes by 32% (P<0.001) while expression of PLTXII reduced eEPSP amplitudes by 96% (P<0.001) indicating the both transgenes were capable of partially inhibiting neurotransmission.

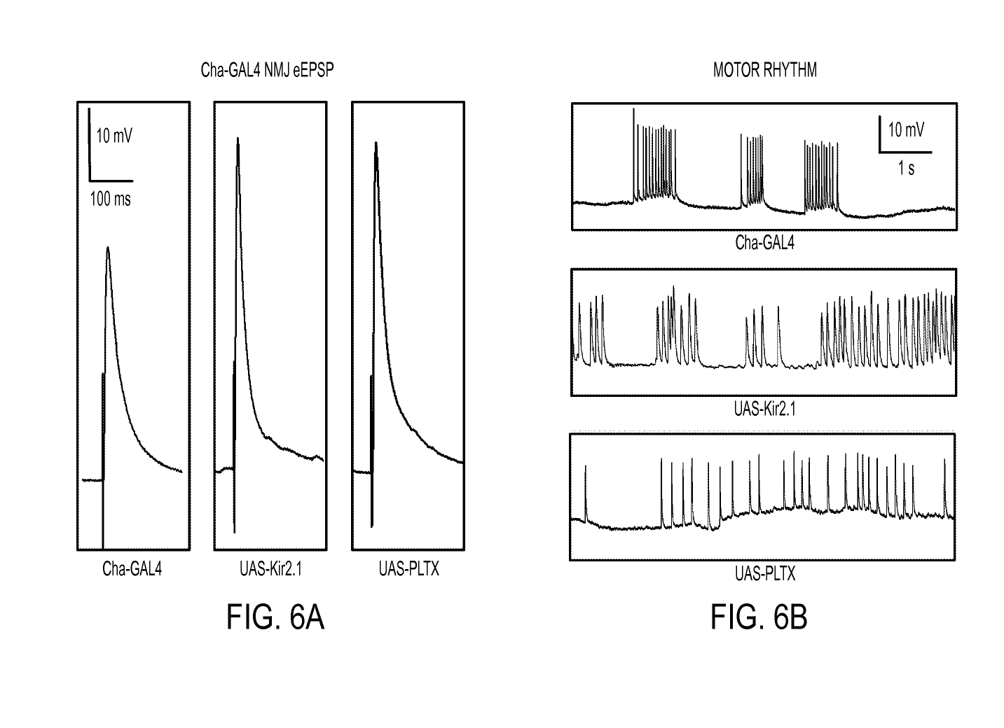

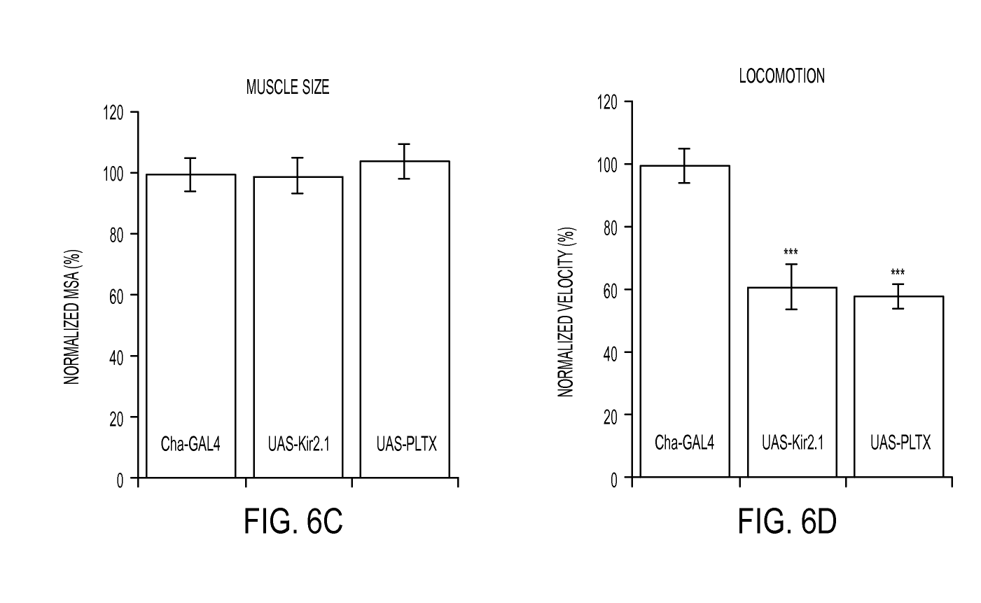

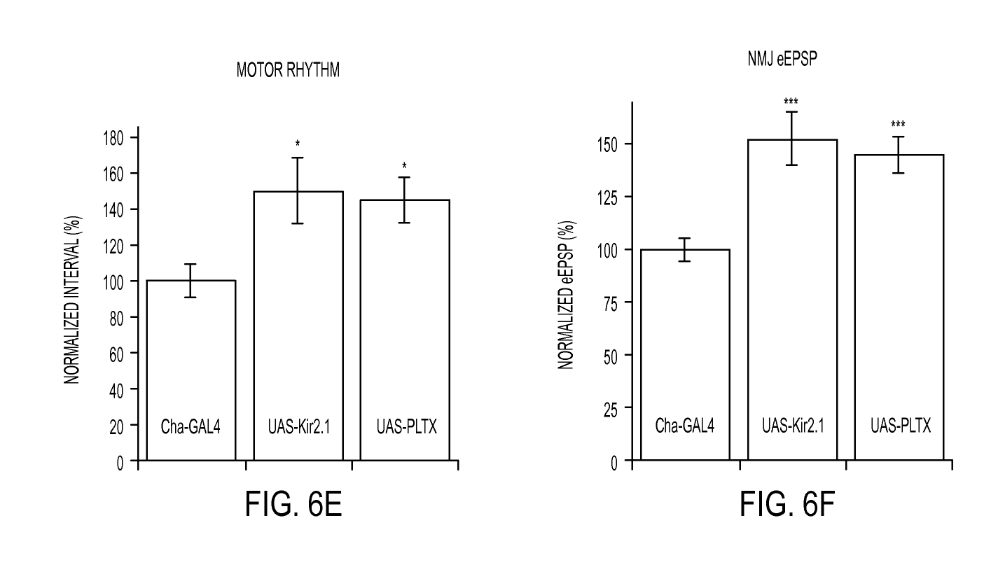

To examine the effects on the motor system of inhibiting cholinergic neuron function, Kir2.1 or PLTXII were expressed in the cholinergic neurons of wild-type animals using Cha-Ga14. Expression of either transgene in cholinergic neurons had no effect on muscle surface area however (FIG. 6C), however expression of these transgenes significantly inhibited locomotion by 41% (P<0.001) and 42% (P<0.001) respectively (FIG. 6D). They also disrupted spontaneous rhythmic motor activity inducing a 54% (P<0.05) or 59% (P<0.05) increase in average inter-spike intervals (FIG. 6B,E). Importantly, inhibition of cholinergic neuron function also resulted in increased eEPSP amplitudes at the NMJs of glutamatergic motor neurons (FIG. 6A,F). Expression of Kir2.1 in choliner.gic neurons produced a 50% increase (P<0.001) in NMJ eEPSP amplitudes while expression of PLTX induced a 45% increase (P<0.001) (FIG. 6F). Therefore, inhibition of cholinergic neuron activity replicated a number of the features of smn mutants including non-cell autonomous effects on the neurotransmitter release properties of motor neurons, consistent with cholinergic neurons in the motor circuit having reduced function in smn mutants.

### Example 4

### Increasing Neuronal Excitability Rescues Smn Mutant Phenotypes

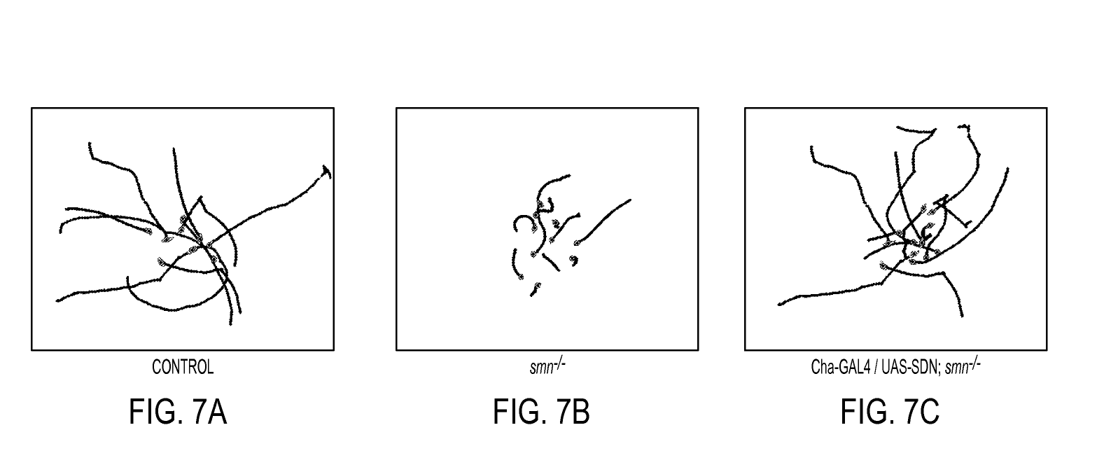

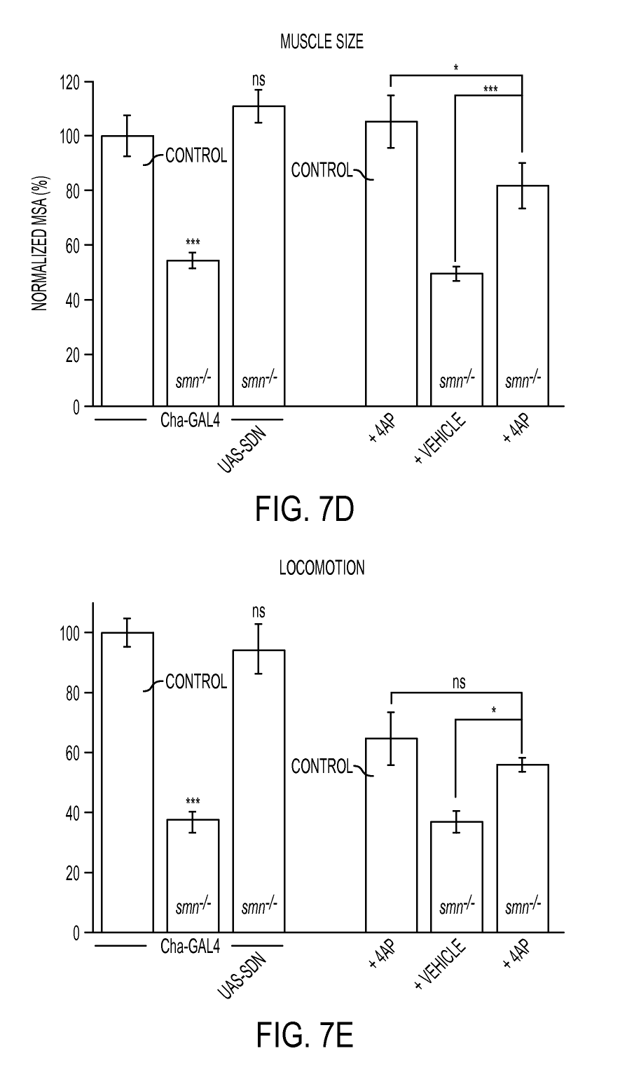

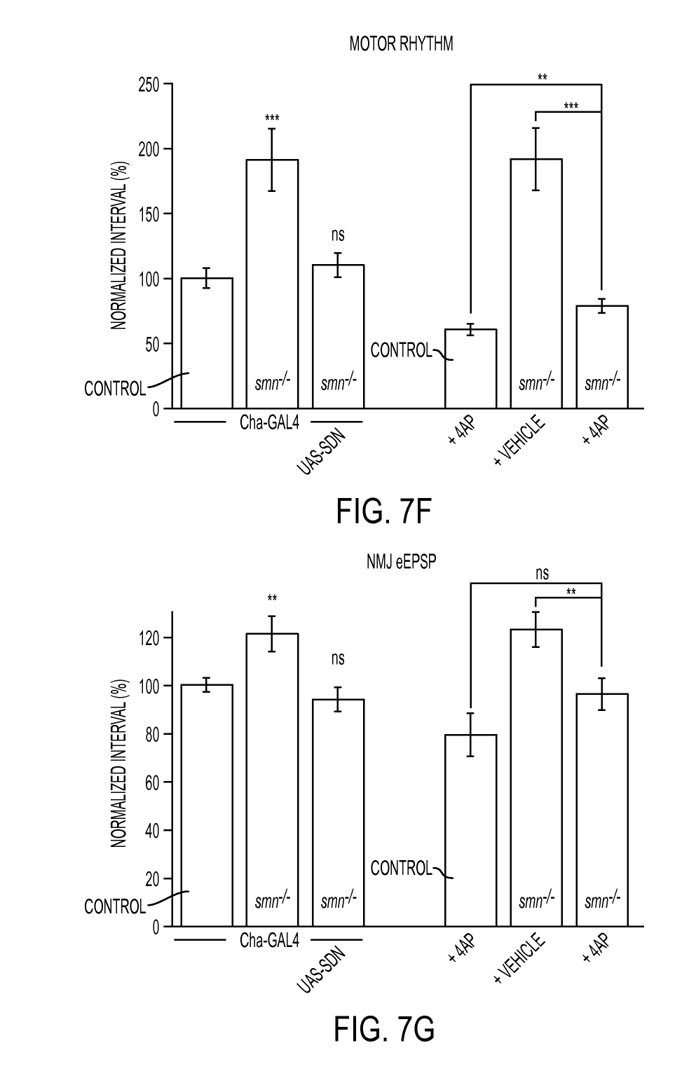

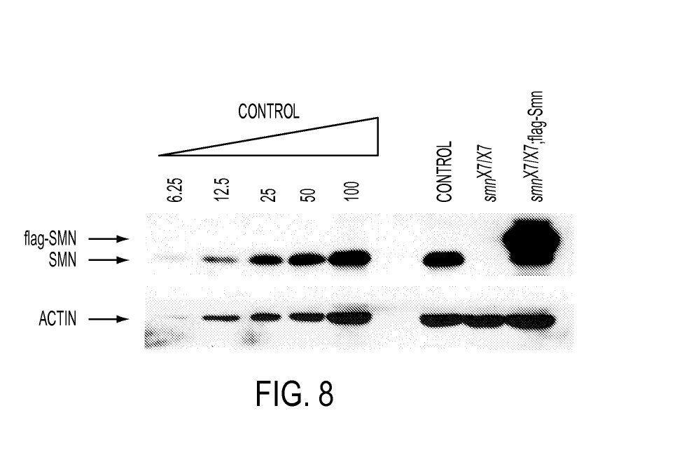

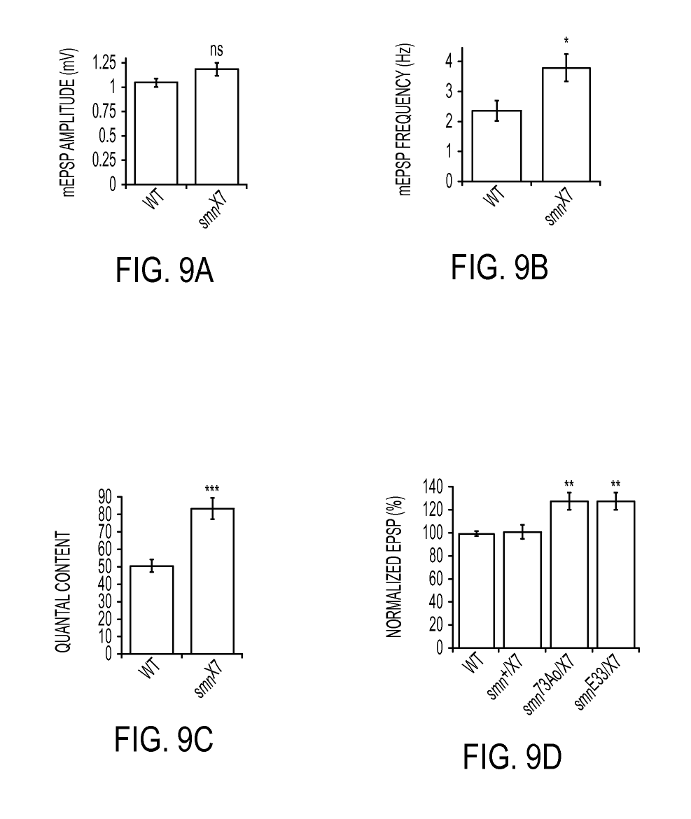

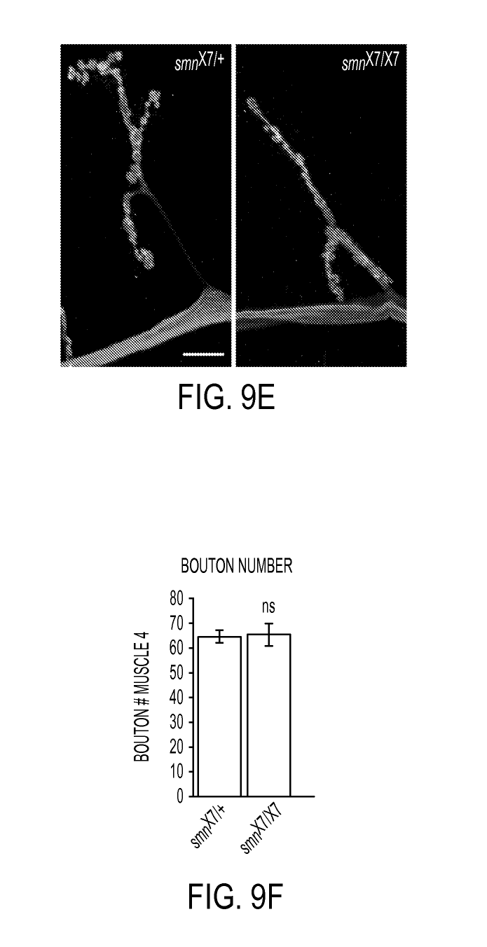

Building upon the hypothesis that motor circuits have functional deficits in smn mutants, experiments were done to determine if increasing the excitability of cholinergic neurons in these animals could increase motor network activity and alter smn mutant phenotypes. Inhibition of the Shaker (Sh) type IA outward K+ current by a dominant negative (SDN) transgene enhances membrane excitability and increases both the amplitude and duration of eEPSPs at synaptic terminals (Mosca et al., 2005). Becuase genetic methods to inhibit K+ channel activity benefited smn mutant phenotypes, experiments were done to test if pharmacological antagonists of K+ channels could also be effective. 4-aminopyridine (4-AP), an FDA approved small molecule inhibitor of voltage activated vertebrate (Hayes, 2007) and Drosophila K+ channels (Wicher et al., 2001) was tested. 4-AP was added to larval media and titrated the compound to identify the maximum dosage at which the drug could be tolerated without lethality in wild-type larvae (2 mM). The effects of exposure of 4-AP throughout the larval period was examined fin both control and smn mutants. In control animals, 4-AP had no effect on muscle size but reduced larval locomotion by 35% (P<0.01), decreased rhythmic motor activity by 40% (P<0.01) and reduced NMJ eEPSP amplitudes by 21% (P<0.001) indicating mild systemic toxicity at this dose (FIG. 7D-G). Despite this, smn mutants were grown on 4-AP containing media throughout the larval period, muscle surface area was increased by 66% (P<0.001) compared to untreated smn mutants (FIG. 7D). Locomotion was also increased by 55% (P<0.05) and was not significantly different to controls treated with 4-AP (FIG. 7E). Defects in rhythmic motor activity in smn mutants were substantially improved with the aberrant increase in inter-spike interval reduced to only 31% (p<0.001) above controls treated with 4-AP (FIG. 7F). Finally, the increased NMJ EPSP amplitude of smn mutants treated with 4-AP were reduced by 27% (P<0.001) not significantly different to that of 4-AP treated controls (FIG. 7G). Thus pharmacological inhibition of K+ channels, similar to genetic inhibition, can positively benefit smn mutant phenotypes consistent with the defective excitability of motor circuits being a critical consequence of SMN depletion.

The invention has been described with reference to specific embodiments. It will, however, be evident that various modifications and changes may be made thereto without departing from the broader spirit and scope of the invention. The specification and drawings are, accordingly, to be regarded in an illustrative rather than a restrictive sense. The invention is illustrated herein by the experiments described above and by the following examples, which should not be construed as limiting. The contents of all references, pending patent applications and published patents, cited throughout this application are hereby expressly incorporated by reference. Although specific terms are employed, they are used as in the art unless otherwise indicated.

TABLE 1
Muscle area, locomotion, motor rhythm, and NMJ ePSP amplitudes

| | Muscle 6 area | Locomotion | Inter-spike | eEPSP |
|---|---|---|---|---|
| Genotype | (×10³ × μm²) | (cm/min) | interval (ms) | amplitude (mV) |
| Canton S | 33.67 ± 3.48 | 4.76 ± 0.22 | 113.72 ± 10.62 | 34.00 ± 1.03 |
| smn^{x7}/smn^{x7} | 18.07 ± 0.91*** | 1.75 ± 0.16*** | 217.17 ± 27.05*** | 41.40 ± 2.42** |
| Actin-Gal4/UAS-SMN; smn^{x7}/smn^{x7} | 33.03 ± 1.75 | 4.45 ± 0.24 | 109.79 ± 7.33 | 32.88 ± 1.03 |
| | ns | ns | ns | ns |
| G14-Gal4; UAS-SMN; smn^{x7}/smn^{x7} | 16.57 ± 1.39*** | 2.06 ± 0.34*** | 188.30 ± 16.85*** | 39.50 ± 1.11*** |
| nsyb-Gal4/UAS-SMN; smn^{x7}/smn^{x7} | 31.71 ± 2.19 | 3.99 ± 0.35 | 98.10 ± 17.66 | 30.10 ± 2.55 |
| | ns | ns | ns | ns |
| Elav-Geneswitch-Gal4/UAS-SMN; | 14.59 ± 1.25*** | 1.87 ± 0.07*** | 235.15 ± 34.82*** | 40.17 ± 0.79** |
| smn^{x7}/smn^{x7}(+ vehicle) | | | | |
| Elav-Geneswitch-Gal4/UAS-SMN; | 30.77 ± 1.62 | 3.83 ± 0.32* | 116.02 ± 12.11 | 32.13 ± 0.93 |
| smn^{x7}/smn^{x7} (RU486 at 0 hours) | ns | | ns | ns |
| Elav-Geneswitch-Gal4/UAS-SMN; | 26.12 ± 2.36** | 3.32 ± 0.37*** | 158.15 ± 8.38* | 34.75 ± 1.69 |
| smn^{x7}/smn^{x7} (RU486 at 48 hours) | | | | ns |
| Elav-Geneswitch-Gal4/UAS-SMN; | 16.19 ± 0.55*** | 1.82 ± 0.11*** | 181.56 ± 33.62** | 33.90 ± 1.34 |
| smn^{x7}/smn^{x7} (RU486 at 96 hours) | | | | ns |
| OK371-Gal4/UAS-SMN; smn^{x7}/smn^{x7} | 17.87 ± 2.71*** | 1.90 ± 0.20*** | 221.00 ± 30.05*** | 40.86 ± 0.88*** |
| OK6-Gal4/UAS-SMN; smn^{x7}/smn^{x7} | 17.79 ± 1.45** | 2.04 ± 0.10*** | 189.6 ± 26.3** | 39.22 ± 1.59*** |
| GAD1-Gal4/UAS-SMN; smn^{x7}/smn^{x7} | 18.22 ± 1.75*** | 1.95 ± 0.09*** | 197.08 ± 32.22** | 43.14 ± 3.02** |
| CHA-Gal4/UAS-SMN; smn^{x7}/smn^{x7} | 32.50 ± 3.89 | 4.10 ± 0.42 | 124.72 ± 10.84 | 32.00 ± 3.24 |
| | ns | ns | ns | ns |
| Clh201-Gal4/UAS-SMN; smn^{x7}/smn^{x7} | 28.12 ± 1.33 | 2.59 ± 0.16 | 146.43 ± 19.88 | 32.73 ± 1.82 |
| | ns | ns | ns | ns |
| 1003.3-Gal4/UAS-SMN; smn^{x7}/smn^{x7} | 20.78 ± 2.02*** | 1.78 ± 0.08*** | 181.17 ± 13.32*** | 40.00 ± 1.15*** |
| ppk-Gal4/UAS-SMN; smn^{x7}/smn^{x7} | 18.38 ± 0.70*** | 1.86 ± 0.10*** | 193.74 ± 26.13** | 33.70 ± 2.11** |
| NP2225-Gal4/UAS-SMN; smn^{x7}/smn^{x7} | 27.69 ± 1.63* | 2.33 ± 0.18*** | 137.66 ± 16.67 | 30.33 ± 1.58 |
| | | | ns | ns |
| Cha-Gal4/UAS-Kir2.1 | 33.32 ± 1.99 | 2.76 ± 0.33*** | 164.95 ± 19.86* | 48.75 ± 4.11*** |
| | ns | | | |
| Cha-Gal4/UAS-PLTXII | 34.99 ± 1.85 | 2.68 ± 0.18*** | 159.44 ± 13.29* | 46.44 ± 2.58*** |
| | ns | | | |
| UAS-SDN/CHA-Gal4; smn^{x7}/smn^{x7} | 37.36 ± 2.24 | 4.50 ± 0.39 | 124.72 ± 10.84 | 30.12 ± 1.88 |
| | ns | ns | ns | |
| CS (treated with 4-AP) | 38.08 ± 3.49 | 3.06 ± 0.40 | 68.89 ± 4.23*** | 26.67 ± 2.91 |
| | ns | | | ns |
| smn^{x7}/smn^{x7} (treated with 4-AP) | 29.60 ± 3.09* | 2.65 ± 0.10 | 89.98 ± 4.98* | 32.40 ± 2.48 |
| | | | | ns |

Muscle area, locomotion, motor rhythm and NMJ eEPSP amplitude measurements for all genotypes.
+/− standard error of the mean. Significance values are calculated versus controls.
*= p < 0.05
**= p < 0.01,
***= p < 0.00

### REFERENCES

Aberle, H., Haghighi, A. P., Fetter, R. D., McCabe, B. D., Magalhaes, T. R., and Goodman, C. S. (2002). wishful thinking encodes a BMP type II receptor that regulates synaptic growth in Drosophila. Neuron 33, 545-558.
Ainsley, J. A., Pettus, J. M., Bosenko, D., Gerstein, C. E., Zinkevich, N., Anderson, M. G., Adams, C. M., Welsh, M. J., and Johnson, W. A. (2003). Enhanced locomotion caused by loss of the Drosophila DEG/ENaC protein Pickpocket1. Curr Biol 13, 1557-1563.
Baines, R. A. (2006). Development of motoneuron electrical properties and motor output. Int Rev Neurobiol 75, 91-103.
Baines, R. A., Uhler, J. P., Thompson, A., Sweeney, S. T., and Bate, M. (2001). Altered electrical properties in Drosophila neurons developing without synaptic transmission. J Neurosci 21, 1523-1531.
Brand, A. H., and Perrimon, N. (1993). Targeted gene expression as a means of altering cell fates and generating dominant phenotypes. Development 118, 401-415.
Brent, J., Werner, K., and McCabe, B. D. (2009a). Drosophila larval NMJ immunohistochemistry. J Vis Exp. 25, 1108.
Brent, J. R., Werner, K. M., and McCabe, B. D. (2009b). Drosophila larval NMJ dissection. J Vis Exp. 24, 1107.
Brent, J. R., Werner, K. M., and McCabe, B. D. (2009). Drosophila larval NMJ dissection. J Vis Exp. 25, 1108.
Burghes, A. H., and Beattie, C. E. (2009). Spinal muscular atrophy: why do low levels of survival motor neuron protein make motor neurons sick? Nat Rev Neurosci 10, 597-609.
Bushey, D., Tononi, G., and Cirelli, C. (2009). The Drosophila fragile X mental retardation gene regulates sleep need. J Neurosci 29, 1948-1961.
Cattaert, D., and Birman, S. (2001). Blockade of the central generator of locomotor rhythm by noncompetitive NMDA receptor antagonists in Drosophila larvae. J Neurobiol 48, 58-73.
Chan, Y. B., Miguel-Aliaga, I., Franks, C., Thomas, N., Trulzsch, B., Sattelle, D. B., Davies, K. E., and van den Heuvel, M. (2003). Neuromuscular defects in a Drosophila survival motor neuron gene mutant. Hum Mol Genet 12, 1367-1376.
Chang, H. C., Dimlich, D. N., Yokokura, T., Mukherjee, A., Kankel, M. W., Sen, A., Sridhar, V., Fulga, T. A., Hart, A. C., Van Vactor, D., et al. (2008). Modeling spinal muscular atrophy in Drosophila. PLoS One 3, e3209.
Chen, H. H., Hippenmeyer, S., Arber, S., and Frank, E. (2003). Development of the monosynaptic stretch reflex circuit. Curr Opin Neurobiol 13, 96-102.
Cheng, L. E., Song, W., Looger, L. L., Jan, L. Y., and Jan, Y. N. (2010). The role of the TRP channel NompC in Drosophila larval and adult locomotion. Neuron 67, 373-380.
Chwieduk, C. M., and Keating, G. M. (2010). Dalfampridine extended release: in multiple sclerosis. CNS Drugs 24, 883-891.
Crawford, T. O., and Pardo, C. A. (1996). The neurobiology of childhood spinal muscular atrophy. Neurobiol Dis 3, 97-110.
Crisp, S., Evers, J. F., Fiala, A., and Bate, M. (2008). The development of motor coordination in Drosophila embryos. Development 135, 3707-3717.
Daniels, R. W., Gelfand, M. V., Collins, C. A., and DiAntonio, A. (2008). Visualizing glutamatergic cell bodies and synapses in Drosophila larval and adult CNS. J Comp Neurol 508, 131-152.
Davis, G. W., DiAntonio, A., Petersen, S. A., and Goodman, C. S. (1998). Postsynaptic PKA controls quantal size and reveals a retrograde signal that regulates presynaptic transmitter release in Drosophila. Neuron 20, 305-315.
Featherstone, D. E., Rushton, E. M., Hilderbrand-Chae, M., Phillips, A. M., Jackson, F. R., and Broadie, K. (2000). Presynaptic glutamic acid decarboxylase is required for induction of the postsynaptic receptor field at a glutamatergic synapse. Neuron 27, 71-84.
Fox, L. E., Soll, D. R., and Wu, C. F. (2006). Coordination and modulation of locomotion pattern generators in Drosophila larvae: effects of altered biogenic amine levels by the tyramine beta hydroxlyase mutation. J Neurosci 26, 1486-1498.
Gavrilina, T. O., McGovern, V. L., Workman, E., Crawford, T. O., Gogliotti, R. G., DiDonato, C. J., Monani, U. R., Morris, G. E., and Burghes, A. H. (2008). Neuronal SMN expression corrects spinal muscular atrophy in severe SMA mice while muscle-specific SMN expression has no phenotypic effect. Hum Mol Genet 17, 1063-1075.
Hayes, K. C. (2007). Fampridine-SR for multiple sclerosis and spinal cord injury. Expert Rev Neurother 7, 453-461.
Hughes, C. L., and Thomas, J. B. (2007). A sensory feedback circuit coordinates muscle activity in Drosophila. Mol Cell Neurosci 35, 383-396.
Ilieva, H., Polymenidou, M., and Cleveland, D. W. (2009). Non-cell autonomous toxicity in neurodegenerative disorders: ALS and beyond. J Cell Biol 187, 761-772.
Imlach, W., and McCabe, B. D. (2009). Electrophysiological methods for recording synaptic potentials from the NMJ of Drosophila larvae. J Vis Exp. 10, 1109
Ito, K., Awano, W., Suzuki, K., Hiromi, Y., and Yamamoto, D. (1997). The Drosophila mushroom body is a quadruple structure of clonal units each of which contains a virtually identical set of neurones and glial cells. Development 124, 761-771.
Jan, L. Y., and Jan, Y. N. (1976). Properties of the larval neuromuscular junction in Drosophila melanogaster. J Physiol 262, 189-214.
Kariya, S., Park, G. H., Maeno-Hikichi, Y., Leykekhman, O., Lutz, C., Arkovitz, M. S.,
Landmesser, L. T., and Monani, U. R. (2008). Reduced SMN protein impairs maturation of the neuromuscular junctions in mouse models of spinal muscular atrophy. Hum Mol Genet 17, 2552-2569.
Kitamoto, T., Xie, X., Wu, C. F., and Salvaterra, P. M. (2000). Isolation and characterization of mutants for the vesicular acetylcholine transporter gene in Drosophila melanogaster. J Neurobiol 42, 161-171.
Kong, L., Wang, X., Choe, D. W., Polley, M., Burnett, B. G., Bosch-Marce, M., Griffin, J. W., Rich, M. M., and Sumner, C. J. (2009). Impaired synaptic vesicle release and immaturity of neuromuscular junctions in spinal muscular atrophy mice. J Neurosci 29, 842-851.
Kuba, H., Oichi, Y., and Ohmori, H. (2010). Presynaptic activity regulates Na(+) channel distribution at the axon initial segment. Nature 465, 1075-1078.
Le, T. T., Pham, L. T., Butchbach, M. E., Zhang, H. L., Monani, U. R., Coovert, D. D., Gavrilina, T. O., Xing, L., Bassell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lefebvre, S., Burglen, L., Reboullet, S., Clermont, O., Burlet, P., Viollet, L., Benichou, B., Cruaud, C., Millasseau, P., Zeviani, M., et al. (1995). Identification and characterization of a spinal muscular atrophy-determining gene. Cell 80, 155-165.
Ling, K. K., Gibbs, R. M., Feng, Z., and Ko, C. P. (2011). Severe neuromuscular denervation of clinically relevant muscles in a mouse model of spinal muscular atrophy. Hum Mol Genet. Ling, K. K., Lin, M. Y., Zingg, B., Feng, Z., and Ko, C. P. (2010). Synaptic defects in the spinal and neuromuscular circuitry in a mouse model of spinal muscular atrophy. PLoS One 5, e15457.
Lorson, C. L., Hahnen, E., Androphy, E. J., and Wirth, B. (1999). A single nucleotide in the SMN gene regulates splicing and is responsible for spinal muscular atrophy. Proc Natl Acad Sci USA 96, 6307-6311.
Mahr, A., and Aberle, H. (2006). The expression pattern of the Drosophila vesicular glutamate transporter: a marker protein for motoneurons and glutamatergic centers in the brain. Gene Expr Patterns 6, 299-309.
Marder, E., and Rehm, K. J. (2005). Development of central pattern generating circuits. Curr Opin Neurobiol 15, 86-93.
Martin, A. R. (1955). A further study of the statistical composition on the end-plate potential. J Physiol 130, 114-122.
McGovern, V. L., Gavrilina, T. O., Beattie, C. E., and Burghes, A. H. (2008). Embryonic motor axon development in the severe SMA mouse. Hum Mol Genet 17, 2900-2909.
Mentis, G. Z., Blivis, D., Liu, W., Drobac, E., Crowder, M. E., Kong, L., Alvarez, F. J., Sumner, C. J., and O'Donovan, M. J. (2011). Early functional impairment of sensory-motor connectivity in a mouse model of spinal muscular atrophy. Neuron 69, 453-467.
Monani, U. R., Lorson, C. L., Parsons, D. W., Prior, T. W., Androphy, E. J., Burghes, A. H., and McPherson, J. D. (1999). A single nucleotide difference that alters splicing patterns distinguishes the SMA gene SMN1 from the copy gene SMN2. Hum Mol Genet 8, 1177-1183.
Mosca, T. J., Carrillo, R. A., White, B. H., and Keshishian, H. (2005). Dissection of synaptic excitability phenotypes by using a dominant-negative Shaker K+ channel subunit. Proc Natl Acad Sci USA 102, 3477-3482.
Ng, M., Roorda, R. D., Lima, S. Q., Zemelman, B. V., Morcillo, P., and Miesenbock, G. (2002). Transmission of olfactory information between three populations of neurons in the antennal lobe of the fly. Neuron 36, 463-474.
Osterwalder, T., Yoon, K. S., White, B. H., and Keshishian, H. (2001). A conditional tissuespecific transgene expression system using inducible GAL4. Proc Natl Acad Sci USA 98, 12596-12601.
Palop, J. J., and Mucke, L. (2010). Amyloid-beta-induced neuronal dysfunction in Alzheimer's disease: from synapses toward neural networks. Nat Neurosci 13, 812-818.
Paradis, S., Sweeney, S. T., and Davis, G. W. (2001). Homeostatic control of presynaptic release is triggered by postsynaptic membrane depolarization. Neuron 30, 737-749.
Park, G. H., Kariya, S., and Monani, U. R. (2010a). Spinal muscular atrophy: new and emerging insights from model mice. Curr Neurol Neurosci Rep 10, 108-117.
Park, G. H., Maeno-Hikichi, Y., Awano, T., Landmesser, L. T., and Monani, U. R. (2010b). Reduced survival of motor neuron (SMN) protein in motor neuronal progenitors functions cell autonomously to cause spinal muscular atrophy in model mice expressing the human centromeric (SMN2) gene. J Neurosci 30, 12005-12019.
Passini, M. A., Bu, J., Roskelley, E. M., Richards, A. M., Sardi, S. P., O'Riordan, C. R., Klinger, K. W., Shihabuddin, L. S., and Cheng, S. H. (2010). CNS-targeted gene therapy improves survival and motor function in a mouse model of spinal muscular atrophy. J Clin Invest 120, 1253-1264.
Pearn, J. (1978). Incidence, prevalence, and gene frequency studies of chronic childhood spinal muscular atrophy. J Med Genet 15, 409-413.
Pellizzoni, L. (2007). Chaperoning ribonucleoprotein biogenesis in health and disease. EMBO Rep 8, 340-345.
Pinter, M. J., Waldeck, R. F., Cope, T. C., and Cork, L. C. (1997). Effects of 4-aminopyridine on muscle and motor unit force in canine motor neuron disease. J Neurosci 17, 4500-4507.
Rajendra, T. K., Gonsalvez, G. B., Walker, M. P., Shpargel, K. B., Salz, H. K., and Matera, A. G. (2007). A Drosophila melanogaster model of spinal muscular atrophy reveals a function for SMN in striated muscle. J Cell Biol 176, 831-841.
Renault, F., Raimbault, J., Praud, J. P., and Laget, P. (1983). [Electromyographic study of 50 cases of Werdnig-Hoffmann disease]. Rev Electroencephalogr Neurophysiol Clin 13, 301-305.
Rothstein, J. D. (2009). Current hypotheses for the underlying biology of amyotrophic lateral sclerosis. Ann Neurol 65 Suppl 1, S3-9.
Salvaterra, P. M., and Kitamoto, T. (2001). Drosophila cholinergic neurons and processes visualized with Gal4/UAS-GFP. Brain Res Gene Expr Patterns 1, 73-82.
Schmid, A., and DiDonato, C. J. (2007). Animal models of spinal muscular atrophy. J Child Neurol 22, 1004-1012.
Simic, G. (2008). Pathogenesis of proximal autosomal recessive spinal muscular atrophy. Acta Neuropathol 116, 223-234.
Song, W., Onishi, M., Jan, L. Y., and Jan, Y. N. (2007). Peripheral multidendritic sensory neurons are necessary for rhythmic locomotion behavior in Drosophila larvae. Proc Natl Acad Sci USA 104, 5199-5204.
Sugimura, K., Yamamoto, M., Niwa, R., Satoh, D., Goto, S., Taniguchi, M., Hayashi, S., and Uemura, T. (2003). Distinct developmental modes and lesion-induced reactions of dendrites of two classes of Drosophila sensory neurons. J Neurosci 23, 3752-3760.
Suster, M. L., and Bate, M. (2002). Embryonic assembly of a central pattern generator without sensory input. Nature 416, 174-178.
Swoboda, K. J., Prior, T. W., Scott, C. B., McNaught, T. P., Wride, M. C., Reyna, S. P., and Bromberg, M. B. (2005). Natural history of denervation in SMA: relation to age, SMN2 copy number, and function. Ann Neurol 57, 704-712.
Wicher, D., Walther, C., and Wicher, C. (2001). Non-synaptic ion channels in insects—basic properties of currents and their modulation in neurons and skeletal muscles. Prog Neurobiol 64, 431-525.
Zlatic, M., Li, F., Strigini, M., Grueber, W., and Bate, M. (2009).

What is claimed is:

1. A method comprising
identifying a subject who has spinal muscular atrophy, and
administering to the subject a therapeutically effective amount of a K+ channel antagonist.

2. The method of claim 1, wherein the wherein the K+ channel antagonist is a broad-based K+ channel antagonist.

3. The method of claim 1, wherein the wherein the K+ channel antagonist is 4-aminopyridine.

4. The method of claim 1, wherein the wherein the K+ channel antagonist is selected from the group consisting of 4-(dimethylamino)pyridine, 4-(methylamino)pyridine, and 4-(aminomethyl)pyridine.

5. The method of claim 1, wherein the wherein the therapeutically effective amount is an amount ranging from about 0.5 mg to 100 mg per administration and the antagonist is administered from one to three times per day.

6. The method of claim 1, wherein the therapeutically effective amount is about 10 mg per administration.

7. The method of claim 1, wherein the K+ channel antagonist is formulated for oral administration.

8. The method of claim 1, wherein the therapeutically effective amount is an amount between about 0.5 mg to 10 mg per administration.

9. A pharmaceutical formulation, comprising 4-AP and one or more agents selected from the group consisting of 4-(dimethylamino)pyridine, 4-(methylamino)pyridine, and 4-(aminomethyl)pyridine.

10. The pharmaceutical formulation of claim 8, wherein the 4-AP and one or more agents are formulated in a liposome for delivery across the blood brain barrier.

11. A pharmaceutical formulation, comprising 4-AP formulated in a liposome for delivery across the blood brain barrier.

12. The method of claim 1, wherein the SMA is type 1 SMA.

13. The method of claim 1, wherein the SMA is type 2 SMA.

14. The method of claim 1, wherein the SMA is type 3 SMA.

15. The method of claim 1, wherein the K+ channel antagonist is administered directly into the epidural venous plexus, brain, spinal column or cerebrospinal fluid.

16. The method of claim 1, wherein the wherein the K+ channel antagonist is selected from the group consisting of dofetilide, sotalol, ibutilide, Azimilide, Bretylium, Clofilium, E-4031, Nifekalant, Tedisamil, and Sematilide.
